(19) United States
(12) Patent Application Publication
(10) Pub. No.: US 2006/0140985 A1
(43) Pub. Date: Jun. 29, 2006

(54) Lansoprazole formulations and related processes and methods

(76) Inventors: Zhong Zhang, Sudbury, MA (US); Orn Almarsson, Shrewsbury, MA (US); Hongming Chen, Accon, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/541,703

(22) PCT Filed: Jan. 8, 2004
(86) PCT No.: PCT/US04/00400

§ 371 Date: Mar. 20, 2006

Related U.S. Application Data
(60) Provisional application No. 60/439,283, filed on Jan. 10, 2003.

(51) Int. Cl. A61K 31/4439; A61K 9/00

(52) U.S. Cl. 424/400; 514/338

(57) ABSTRACT

The invention provides novel liquid lansoprazole formulations comprising lansoprazole and an excipient system, wherein: (a) the concentration of lansoprazole in the formulations ranges from about 0.3 mg/mL to about 50 mg/mL; (b) the excipient system comprises either a single excipient, or a combination of two to four compositionally distinct excipients; and (c) the formulations may be administered parenterally to a mammal to treat or prevent a gastrointestinal disorder.

### RELATED APPLICATIONS

This application claims the benefit of U.S. Provisional Application No. 60/439,283, filed Jan. 10, 2003.

### FIELD OF THE INVENTION

The invention provides novel liquid lansoprazole formulations and related excipient systems comprising lansoprazole, or a derivative, analog, or salt of lansoprazole, and an excipient system, wherein: (a) the concentration of lansoprazole, or the derivative, analog, or salt of lansoprazole, in the formulations ranges from about 0.3 mg/mL to about 50 mg/mL; (b) the excipient system comprises either a single excipient, or a combination of two or more compositionally distinct excipients; and (c) the formulation may be administered parenterally to a mammal to treat or prevent a gastrointestinal disorder.

### BACKGROUND OF THE INVENTION

Lansoprazole, 2-[[[3-methyl-4 (2,2,2-trifluoroethoxy)-2-pyridyl]methyl]sulfinyl]benzimidazole (marketed in the United States under the trademark PREVACID®), is a substituted benzimidazole that inhibits gastric acid secretion. The empirical formula of lansoprazole is C₁₆H₁₄F₃N₃O₂S and the compound has a molecular weight of 369.37. The structural formula of lansoprazole is (I):

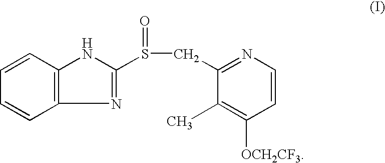

Lansoprazole is a white to brownish-white odorless crystalline powder which melts with decomposition at approximately 180 degrees C. Lansoprazole is freely soluble in dimethylformamide, soluble in methanol, sparingly soluble in ethanol, slightly soluble in ethyl acetate, dichloromethane and acetonitrile, very slightly soluble in ether, and is practically insoluble in hexane and water. Lansoprazole is stable when exposed to light for up to two months. The compound degrades in aqueous solution, with the rate of degradation increasing with decreasing pH.

Lansoprazole is extensively metabolized in the liver. Two metabolites have been identified in measurable quantities in plasma (the hydroxylated sulfinyl and sulfone derivatives of lansoprazole). These metabolites have very little or no antisecretory activity. Lansoprazole is thought to be transformed into two active species which inhibit acid secretion by (H⁺K⁺)-ATPase within the parietal cell canaliculus, but which are not present in the systemic circulation. The plasma elimination half-life of lansoprazole does not reflect its duration of suppression of gastric acid secretion. Thus, the plasma elimination half-life is less than two hours, while the acid inhibitory effect lasts more than twenty-four hours.

Lansoprazole belongs to a class of antisecretory compounds called proton pump inhibitors (“PPIs”) that do not exhibit anti-cholinergic or H₂ histamine antagonist properties. Typically, lansoprazole and other PPI's are formulated in an enteric-coated solid dosage form (as either a delayed-release capsule or tablet), and are prescribed for short-term treatment of active duodenal ulcers, gastric ulcers, gastroesophageal reflux disease (GERD), severe erosive esophagitis, poorly responsive systematic GERD, and pathological hypersecretory conditions such as Zollinger Ellison syndrome. These conditions are caused by an imbalance between acid and pepsin production, called aggressive factors, and mucous, bicarbonate, and prostaglandin production, called defensive factors. These above-listed conditions commonly arise in healthy or critically ill patients, and may be accompanied by significant upper gastrointestinal bleeding.

U.S. Pat. No. 6,489,346 (“'346 Patent”) describes the disadvantages attendant to administration of known PPI formulations to certain categories of patients. For example, it can be difficult or impossible to administer an oral dosage form of a PPI parenterally to critically ill patients, children, the elderly, and patients suffering from dysphagia; they may be either-unwilling or unable to swallow tablets or capsules. The PPI formulations described in the '346 Patent suffer from numerous drawbacks, including use of undesirable formulation agents such as sodium bicarbonate, difficulty in administration, and large active ingredient dosage size. The current unavailability of a stable and versatile parenteral lansoprazole formulation poses a particular clinical drawback in the treatment of the critically ill and elderly.

Parenteral compositions are introduced into an organism or host by means other than through the gastrointestinal tract. Particularly, parenteral formulations are introduced into a host by subcutaneous (SC) or intramuscular (IM) injection or infusion. Injectable suspensions may be formulated as a ready-to-use injection or require a reconstitution step prior to use. Parenteral formulations are frequently administered through needles about one-half to two inches long, 19 to 22 gauge, with an internal diameter in the range of 700 to 400 microns, respectively.

While there is a great need for lansoprazole formulations that can be administered parenterally, it is difficult in general to make a safe and effective parenteral (injectable) formulation. To be effective and pharmaceutically acceptable, injectable formulations should preferably be: sterile; stable; resuspendable; syringeable; injectable; isotonic; and nonirritating. The foregoing characteristics result in manufacturing, storage, and usage requirements that make injectable suspensions one of the most difficult dosage forms to develop.

The need exists, therefore, for stable and versatile liquid lansoprazole formulations that may be administered parenterally to patients who cannot or will not tolerate oral administration of lansoprazole. The need also exists for excipient systems that will increase the parenteral bioavailability of lansoprazole at desired dosage ranges and ensure long-term formulation stability.

### SUMMARY OF THE INVENTION

The invention encompasses lansoprazole formulations and related excipient systems which may be administered parenterally, are stable during storage, and exhibit increased bioavailability. The formulations and excipient systems of the invention are useful for parenterally administering lansoprazole, its derivatives, or pharmaceutically acceptable salts of such derivatives to patients in need thereof. The formulations and excipient systems of the invention are particularly useful for increasing the parenteral bioavailability of lansoprazole, such that the parenteral route is a useful route of administration. The formulations and excipient systems are chemically and physically stable over a wide range of environmental conditions.

Typically, the formulations and excipient systems of the present invention maintain the solvation or suspension of lansoprazole and its derivatives or salts over long periods of time and under conditions more unfavorable to thermodynamic stability (e.g., at higher temperatures). For example, the compositions exhibit naked eye visual clarity at room temperature over extended periods of time of as long as eight to ten hours.

More generally, the formulations and excipient systems of the invention or mixtures thereof can increase the solubility of lansoprazole and derivatives thereof as well as its systemic bioavailability such that the invention encompasses a parenteral formulation which can be administered to a wide variety of patients.

In one embodiment, the invention includes a liquid pharmaceutical formulation suitable for parenteral administration to a mammal comprising:
(a) lansoprazole, a derivative, or a pharmaceutically acceptable salt thereof; and
(b) one or more of an oil, a solvent, a surfactant or another excipient, each of which is defined further below.

In another embodiment, the invention includes a liquid pharmaceutical formulation suitable for parenteral administration to a mammal comprising:
(a) lansoprazole, a derivative, or a pharmaceutically acceptable salt thereof; and
(b) an oil, a solvent, a surfactant, or another excipient.

More specifically, in one embodiment, the instant invention provides novel liquid lansoprazole formulations comprising lansoprazole and an excipient system, wherein:
(a) the concentration of lansoprazole in the formulations ranges from about 0.3 mg/mL to about 50 mg/mL;
(b) the excipient system comprises either a single excipient, or a combination of two to four compositionally distinct excipients, wherein each excipient is selected from the group of excipient categories consisting of: a hydrotrope, a preservative, a pharmaceutically acceptable salt, a surfactant, a base, a cyclodextrin, a viscosity modifier, an emulsifier, a solvent, a carrier, and a lubricant; and
(c) the formulation may be administered parenterally.

The invention encompasses parenteral lansoprazole formulations which have higher solubilities of lansoprazole when compared to previous attempts to make parenteral lansoprazole. The formulations of the invention provide for a higher concentration of lansoprazole while lowering the overall volume of the dose of lansoprazole needed for therapeutic effect. Importantly, the excipient systems used in the formulations of the instant invention should prove well-tolerated by patients.

The invention also encompasses processes for preparing parenteral formulations of lansoprazole or derivatives thereof. In one process, the formulations of the invention are prepared by dissolving lansoprazole, a derivative or a pharmaceutically acceptable salt thereof, in a solvent of the invention prior to dilution with one or more lansoprazole-free oils, solvents, surfactants or other excipients as described herein. Such a method increases the amount of lansoprazole that can be formulated and thus delivered parenterally. In a preferred injectable embodiment, the concentration of lansoprazole after dilution with one or more lansoprazole-free oils, solvents, surfactants or other excipient is 4.0 mg/mL or greater. In another preferred infusion embodiment, the concentration of lansoprazole after dilution with one or more lansoprazole-free oils, solvents, surfactants or other excipient is 0.4 mg/mL or greater.

The invention further encompasses methods of parenterally delivering lansoprazole to a mammal which comprises administering lansoprazole within one of the formulations of the invention. In a preferred embodiment, the invention encompasses methods of augmenting the bioavailability of lansoprazole in a mammal; more preferably increasing the systemic bioavailability of lansoprazole in a mammal.

Formulations of the instant invention may be administered parenterally to a mammal to treat or prevent a gastrointestinal disorder, as defined hereinafter. Significantly, the formulations prove particularly efficacious in administering lansoprazole to humans who are: unable to tolerate oral dosages of lansoprazole for short-term treatment (e.g., up to five days); critically ill; children; elderly; or suffering from dysphagia, reflux oesophagitis, duodenal and benign gastric ulcers, or complications from NSAID therapy.

More specifically, formulations of the invention having a lansoprazole concentration of about 0.3 mg/mL to 0.4 mg/mL or greater or may be administered to a mammal by continuous infusion over a period of between about ten to twenty minutes to treat or prevent a gastrointestinal disorder. Alternatively, formulations of the invention having a lansoprazole concentration of about 4 mg/mL or greater may be administered to a mammal by injection over a period of ten minutes or less to treat or prevent a gastrointestinal disorder.

Particularly preferred excipients useful in formulations of the instant invention are defined hereinafter and include sorbitol, mannitol, lactose, benzethonium chloride, chlorobutanol, methylparaben, methanol, ethanol, i-propanol, n-butanol, calcium chloride, magnesium chloride, calcium gluconate, calcium glubionate, calcium gluceptate, polyoxyethylated castor oil, polysorbate 20 (Tween 20), polysorbate 80 (Tween 80), poloxamers, deoxycholic acid and salts of deoxycholic acid, lysine, diethanolamine, gamma cyclodextrin, hydroxypropyl-beta-cyclodextrin, polyvinylpyrrolidone, glycerin, lecithin, sodium benzoate, sodium acetate, sodium tartrate, polyethylene glycol, starch, and propylene glycol.

In preferred embodiments, the concentrations of each excipient in the formulations of the instant invention can range from about 0.3 mg/mL to about 30 mg/mL and total excipient concentration in the formulations ranges from about 0.6 mg/mL to about 60 mg/mL. Diluents and preservatives may be added to the formulations as necessary.

In preferred embodiments, formulations of the instant invention comprise lansoprazole and a single excipient, or lansoprazole in combination with a two-excipient system comprising either compositionally distinct first and second excipients selected from the same excipient category as defined hereinafter, or compositionally distinct first and second excipients selected from different excipient categories. In still another preferred embodiment, the formulations comprise three or four excipients selected from the same or different excipient categories. The invention includes processes for making these formulations.

These and other aspects of the invention are described further in the following detailed description.

### DETAILED DESCRIPTION OF THE INVENTION

As used herein, the following terms have the following respective meanings.

The term “mammal” as used herein, encompasses any mammal. Preferably, a mammal is in need of a formulation of the invention. Examples of mammals include, but are not limited to, cows, horses, sheep, pigs, cats, dogs, mice, rats, rabbits, guinea pigs, monkeys, etc., more preferably, a human. In certain embodiments, the mammal is an infant, child, adolescent, or adult.

“Derivatives and analogs of lansoprazole” include, as disclosed in U.S. Pat. No. 4,628,098, the complete disclosure of which is hereby incorporated by reference, compounds having the general formula (II) below and stereoisomers and pharmaceutically acceptable salts thereof: 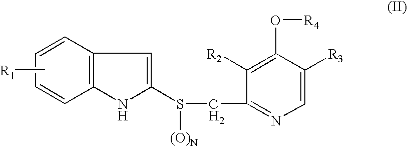
wherein, in one example, R₁ is hydrogen, methoxy or trifluoromethyl, R₂ and R₃ are independently hydrogen or methyl, R₄ is a C₂₋₅ fluorinated alkyl, and n is 0 or 1. “Pharmaceutically acceptable salts of lansoprazole” means those salts of lansoprazole derivatives that retain the biological effectiveness and properties of the free acids or free bases and that are not otherwise unacceptable for pharmaceutical use. Pharmaceutically acceptable salts of lansoprazole derivatives include salts of acidic or basic groups which may be present in the lansoprazole derivatives. Derivatives of lansoprazole that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, such as chloride, bromide, iodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Derivatives of lansoprazole that include an amino moiety can also form pharmaceutically acceptable salts with various amino acids, in addition to the acids mentioned above. Derivatives of lansoprazole that are acidic in nature are capable of forming a wide variety of salts with various inorganic and organic bases. Suitable base salts are formed from bases that donate cations to form non-toxic salts, suitable cations include, but are not limited to, sodium, aluminum, calcium, lithium, magnesium, potassium, zinc and diethanolamine salts. For a review on pharmaceutically acceptable salts see Berge et al., J. Pharm. Sci., 66, 1-19 (1977), incorporated herein by reference.

“Excipient” refers to the substances used to formulate active pharmaceutical ingredients (APIs) into pharmaceutical formulations; in a preferred embodiment, an excipient does not lower or interfere with the primary therapeutic effect of the active. Preferably, an excipient is therapeutically inert. Excipients can also be those substances present in a pharmaceutical formulation as an indirect result of the manufacturing process. Preferably, excipients are approved for or considered to be safe for human and animal administration, i.e., GRAS substances (generally regarded as safe). GRAS substances are listed by the Food and Drug administration in the Code of Federal Regulations (CFR) at 21 CFR 182 and 21 CFR 184, which is incorporated herein by reference.

The following preferred excipient groupings comprise sets of illustrative excipients that may be combined in making excipient systems of the instant invention. There may be overlap between the members of each grouping. It is to be understood that the specific excipients listed herein are merely representative of excipients within a particular excipient category. Those skilled in the art will appreciate that the excipients exemplified herein are merely illustrative of the wide variety of excipient types that may be used to make the formulations and excipient systems of the instant invention. Additional suitable excipients that may be used can be found in references including the Handbook of Pharmaceutical Additives compiled by Michael and Irene Ash, Gower Publishing, 1995, which is incorporated herein by reference in its entirety.

In a first grouping, excipients useful in the instant invention may be chosen from the following excipients known to be useful as suitable for injection into an animal, in particular, a human. These include ammonium acetate, benzalkonium chloride, benzethonium chloride, benzyl alcohol, Brij 35, Brij 97, calcium gluceptate, chlorobutanol, polyoxyethylated castor oil, deoxycholate, diethanolamine, ethanol, gamma cyclodextrin, glycerin, lactobionic acid, lysine, magnesium chloride, methylparaben, PEG 1000, PEG 300, PEG 3350, PEG 400, PEG 600, polyethylene glycol 40 stearate (i.e., PEG 40 stearate), poloxamer 188, poloxamer 237, poloxamer 338, poloxmer 407, polyoxyethylene 100 stearate, polyoxyethylene 40 stearate, polyoxyethylene 50 stearate, polysorbate 20 TWEEN 20, TWEEN is a trademark of ICI Americas, Inc.), polysorbate 80 (i.e., TWEEN 80, TWEEN is a trademark of ICI Americas, Inc.), povidone, propylene glycol, saccharin sodium, sodium acetate, sodium deoxycholate, vitamine E T?GS, sodium benzoate, and sodium tartrate.

In a second grouping, excipients useful in the instant invention may be chosen from the following excipients and excipient combinations:
ammonium acetate and poloxamer;
ammonium acetate, polyoxyethylene, and poloxamer;
lauryl ether, benzyl alcohol, and poloxamer;
polyoxyethylene, lauryl ether or polyoxyethylene, oleyl ether and poloxamer;
polyoxyethylene, lauryl ether or polyoxyethylene, oleyl ether and polysorbate 20 or polysorbate 80;
combinations of polyoxyethylene, lauryl ether, polyoxyethylene, oleyl ether, D-alpha-tocopheryl polyethylene glycol 1000 succinate, polyoxyethylene lauryl ether, and polyoxyethylene oleyl ether;
chlorobutanol and poloxamer;
polyoxyethylated castor oil and poloxamer;
polyoxyethylated castor oil and polysorbate;
polyoxyethylated castor oil and polyoxyethylene stearates;
polyoxyethylated castor oil and propylene glycol;
polyoxyethylated castor oil and D-alpha-tocopheryl polyethylene glycol 1000
succinate; deoxycholate and D-alpha-tocopheryl polyethylene glycol 1000
succinate; deoxycholate and poloxamer;
diethanolamine and D-alpha-tocopheryl polyethylene glycol 1000 succinate;
diethanolamine and poloxamer;
ethanol and D-alpha-tocopheryl polyethylene glycol 1000 succinate;
glycerin and poloxamer;
glycerin and D-alpha-tocopheryl polyethylene glycol 1000 succinate;
lactobionic acid and poloxamer; lysine and poloxamer;
magnesium chloride and poloxamer;
polyethylene glycol and poloxamer;
polyethylene glycol and D-alpha-tocopheryl polyethylene glycol 1000 succinate;
polyethylene glycol and polysorbate; polyoxyethylene stearates and polysorbate;
polyoxyethylene stearates and D-alpha-tocopheryl polyethylene glycol 1000
succinate; polysorbate and D-alpha-tocopheryl polyethylene glycol 1000
succinate;
polysorbate and propylene glycol; and
D-alpha-tocopheryl polyethylene glycol 1000 succinate and propylene glycol.

In a third grouping, excipients useful in the instant invention may be chosen from one or more members of eleven “Preferred Excipient Categories”. The eleven “Preferred Excipient Categories” are hydrotropes, preservatives, pharmaceutically acceptable salts, surfactants, bases, cyclodextrins, viscosity modifiers, emulsifiers, solvents, carriers, and lubricants. Examples of the members of the “Preferred Excipient Categories” include sorbitol, mannitol, lactose, benzethonium chloride, chlorobutanol, methylparaben, methanol, ethanol, i-propanol, n-butanol, calcium chloride, magnesium chloride, calcium gluconate, calcium glubionate, calcium gluceptate, polyoxyethylated castor oil, polysorbate 20 (Tween 20), polysorbate 80 (Tween 80), poloxamers, deoxycholic acid and salts of deoxycholic acid, lysine, diethanolamine, gamma cyclodextrin, hydroxypropyl-beta-cyclodextrin, polyvinylpyrrolidone, glycerin, lecithin, sodium benzoate, sodium acetate, sodium tartrate, polyethylene glycol, starch, and propylene glycol. It is to be understood that the specific excipients listed herein are merely representative of excipients within a particular excipient category and that an individual excipient may fall within more than one excipient category.

The eleven “Preferred Excipient Categories” are further defined as follows.

“Hydrotropes” are compounds that that have the ability to increase the solubility of slightly soluble organic compounds. Hydrotropes useful in the present invention include sorbitol, mannitol, nicotinic acid, nicotinamide, 2,5-dihydroxybenzoic acid, ascorbic acid, ascorbyl dipalmitate, fructose, glucose, glucose glutamate, glucuronic acid, glycerin, 1,2,6-hexanetriol, hydroxystearyl methylglucamine, inositol, lactose, maltitol, sorbeth-20, sucrose, thioglycerin, tris(hydroxymethyl)nitromethane, tromethamine and xylitol, and polyhydroxylated alcohols including sorbitol. Preferred hydrotropes include sorbitol, mannitol, and lactose.

“Preservatives” useful in the formulations of the instant invention include benzalkonium chloride, benzethonium chloride, chlorobutanol, phenylethyl alcohol, thimerosal, methylparaben, propylparaben, and benzyl alcohol and combinations thereof. Benzethonium chloride and methylparaben are preferred preservatives.

“Pharmaceutically acceptable salts” as used herein preferably include salts of sodium, potassium, lithium, magnesium, iron, zinc, including acetate, citrate, benzoate, tartrate and malate salts. “Pharmaceutically acceptable salts” also include salts of calcium, such as calcium chloride, calcium gluconate, calcium glubionate or calcium gluceptate. Sodium acetate, sodium benzoate, sodium tartate, magnesium chloride, and calcium gluceptate are especially preferred salts.

“Surfactants” and “a surfactant of the invention” refer to a compound including, but not limited to, polyoxyl 20 stearate, polyoxyl 35 castor oil, poloxamer, polyoxyethylene sorbitan monoisostearate, polyethylene glycol 40 sorbitan diisostearate, polyoxyl 40 hydrogenated castor oil, polysorbate, polysorbate 20, polysorbate 40, polyoxyl 60 stearate, polysorbate 85, polysorbate 60, poloxamer 331, polyoxyethylene fatty acid esters, polyoxyl 40 castor oil, poloxamer 188, polyoxyethylene polyoxypropylene 1800, oleic acid, sodium desoxycholate, sodium lauryl sulfate, sorbitan monolaurate, sorbitan monooleate, sorbitan monopalmitate, sorbitan trioleate, N-carbamoyl methoxypolyethylene glycol 2000-1,2-distearol, myristic acid, steareth, stearic acid, polyoxyl 40 stearate, sucrose stearate, tocopherol, polyoxyl castor oil, triglyceride synthetic, trimyristin, tristearin, magnesium stearate, lecithin, lauryl sulfate, vitamin E, egg yolk phosphatides, docusate sodium, polysorbate 80, dimyristoyl phosphatidylglycerol, dimyristoyl lecithin, Capryol 90 (propylene glycol monocaprylate), Capryol PGMC (propylene glycol monocaprylate), deoxycholate, cholesterol, polyoxyethylated castor oil, propylene glycol alginate, Croval A-10 (PEG 60 almond glycerides), Labrafil 1944 (oleoyl macrogol-6 glycerides), Labrafil 2125 (linoleoyl macrogol-6 glycerides), Labrasol (caprylocaproyl macrogol-8 glycerides), Lauroglycol 90 (propylene glycol monolaurate), Lauroglycol FCC (propylene glycol laurate), calcium stearate, lecithin Centromix E, Lecithin Centrophase 152, Lecithin Centrol 3F21B, POE 26 glycerin, Olepal isosteariques (PEG-6 isostearate), Plurol diisostearique (polyglycerol-3-diisostearate), Plurol Oleique CC, POE 20 sorbitan trioleate, Tagat TO (polyoxyethylene glycerol trioleate), or Solutol (Macrogol-15 hydroxystearate).

Preferred surfactants include polysorbate 80 and polyoxyethylene 20 sorbitan monoleate, polyoxyxethylene alkyl ethers of the Brig- or Volpo series, polyoxyethylated castor oil , polyoxyethylene sorbitant fatty acid esters of the Tween- or Crillet series, polyoxyethylene stearates of the Cerosynt- or Myrj series, lecithin, poloxamers, d-2-tocophenyl polyethylene glycol 1000 succinate (Vitamine E TPGS) Poloxamer and saturated polyglycolized glycerides (Labrosol, Labrafile and Gelucires), cholic acid and salts of cholic acid, deoxycholic acid and salts of deoxycholic acid, taurocholic acid, salts of taurocholic acid, polyvinylpyrrolidone, cocamines, glyceryl stearates, glyceryl oleates, hydrogenated lanolins, lanolins, laurates and oleates, sorbitan laurates, sorbitan palmitates, sorbitan stearates, quaternium surfactants, sodium sulfates, glyceryl compounds, palmitic acid and its derivatives and oleic acid and its derivatives. Especially preferred surfactants include sodium deoxycholate, sodium taurocholate, TWEEN 20, TWEEN 80, and sodium dodecylsulfate.

“Bases” useful in the formulations of the instant invention include arginine, lysine, cyclohexylamine, ethanolamine, diethanolamine and triethanolamine, sodium hydroxide, and potassium hydroxide. Diethanolamine is a preferred base.

“Cyclodextrins” useful in the formulations of the instant invention include alpha cyclodextrin, beta cyclodextrin, gamma cyclodextrin, or alkyl or hydroxy alkyl derivatives thereof. Cyclodextrins also include hydroxyethyl, glucosyl, maltosyl and maltotriosyl derivatives of beta-cyclodextrin and the corresponding derivatives of gamma-cyclodextrin. The hydroxyalkyl groupings may contain one or more hydroxyl groups, e.g., hydroxypropyl (2-hydroxypropyl, 3-hydroxypropyl), dihydroxypropyl and the like. The glucosyl, maltosyl and maltotriosyl derivatives may contain one or more sugar residues, e.g. glucosyl or diglucosyl, maltosyl or dimaltosyl. Various mixtures of the cyclodextrin derivatives may be used as well, e.g., a mixture of maltosyl and dimaltosyl derivatives. Preferred cyclodextrins include beta cyclodextrin, gamma cyclodextrin, hydroxypropyl beta cyclodextrin, hydroxypropyl-beta-cyclodextrin (HPCD or HPBCD) and randomly methylated beta cyclodextrin.

“Viscosity modifiers” useful in the formulations of the instant invention include polyvinyl alcohol, cellulose derivatives, polyvinylpyrrolidone, polysorbates, and glycerin. Polyvinylpyrrolidone is a preferred viscosity modifier.

“Emulsifiers” useful in the formulations of the instant invention include lecithin, sorbitan monooleate, and acacia. Lecithin is a preferred emulsifier.

“Solvents” useful in the formulations of the instant invention include, but are not limited to, alcohols of the formula ROH, where R is a straight or branched chain, substituted or unsubstituted, lower alkyl (preferred examples of these alcohols include methanol, ethanol, i-propanol and n-butanol) cetyl alcohol, glyceryl stearate, isopropyl alcohol, diethylamine, ethylene glycol monoethyl ether, Transcutol, benzyl alcohol, glyceryl oleate, gelucire, myristyl alcohol, diethanolamine, glycerin, glyceryl distearate, gamma cyclodextrin, gelatin, ethylene glycol, polyethylene glycol 8000, Cresol, Propylene glycol, polyethylene glycol, polyethylene glycol 1000, polyethylene glycol 1450, polyethylene glycol 1540, polyethylene glycol 200, polyethylene glycol 300, polyethylene glycol 3350, polyethylene glycol 3500, polyvinylpyrrolidone, polyethylene glycol 400, polyethylene glycol 4000, polyethylene glycol 600, polyethylene glycol 6000, stearyl alcohol, polyethylene glycol t-dodecylthioehter, polyethylene oxide, triacetin, polyvinylpyridine, polyvinyl alcohol, polypropylene glycol, or Arlacel 186 (monoolein:propylene glycol=90: 10).

Preferred solvents include diethylene glycol monoethyl ether, propylene carbonate, dimethyl isosorbide, -I-methyl-2-pyrrolidinone (NMP), medium chain length monoglycerides, a polyol, Transcutol® (a highly purified diethylene glycol monoethyl ether), medium chain length monoglycerides including glyceryl monocaprylate (Imwitorg.), glyceryl caprylate/caprate (such as Capmul®), polyoxyethylene glyceryl caproate (such as Labrasol®). Polyols which may be used as solvents include glycerin, propylene glycol, 1,4-butane diol, 1,3-butane diol, hexylene glycol, tetraglycol (glycofuranol), or polyethylene glycol. Preferred polyols include polyethylene glycols or “PEGs”, which refer to a liquid or solid polymer of the general formula H(OCH₂ CH₂),OH, wherein n is at least 4. A preferred PEG has an average molecular weight of from about 200 to about 5000 Daltons, or from about 300 to about 2000 Daltons, or from about 300 to about 1500 Daltons. Commercially available PEG materials include PEG 12, PEG-200, PEG-300, PEG-400, PEG-540, PEG-600, PEG-800, PEG-1000 and PEG-1450. PEG 300, PEG 400, and PEG 600 are especially preferred solvents.

“Carriers” useful in the formulations of the instant invention include calcium carbonate, calcium phosphate, calcium crospovidone sulphate, microcrystalline cellulose, cellulose dextrates, dextrin, dextrose excipient, fructose, lactose, mannitol, sorbitol, starch povidone, pregelatinized starch, sucrose, compressible sugar or confectioner's sugar. Carriers can also include “lubricants” as defined herein. Lactose is a preferred carrier.

“Lubricants” used in these dosage forms include sodium oleate, sodium stearate, magnesium stearate, sodium benzoate, sodium acetate, sodium chloride, calcium stearate, polyethylene glycol, stearic acid, talc, zinc stearate, gum acacia, calcium phosphate, alginates, tragacanth, gelatin, calcium silicate, microcrystalline cellulose, calcium stearate, magnesium stearate, polyvinylpyrrolidone, cellulose, water, syrup and methyl cellulose, fatty acids such as oleic acid and its glyceride derivatives, and natural pharmaceutically-acceptable oils such as olive oil or castor oil (especially in their polyoxyethylated versions). Polyvinylpyrrolidone is a preferred lubricant.

“Oil” and “oil of the invention” refer to an excipient useful in the instant invention that includes, but is not limited to, Myverol 18-92, acetylated monoglycerides, Alkamuls 719, Alkamuls 620, Miglyol 812 (caprylic/capric triglyceride), canola oil, caprylic/capric triglyceride, cassia oil, castor oil, castor oil hydrogenated, palm oil, hydrogenated soybean oil, Captex 335 (C8/C10 triglycerides from coconut oil), corn glycerides, corn oil, corn oil PEG-6 esters, cottonseed oil, Captex 200 (C8/C10 diesters of propylene glycol of coconut oil), diacetylated monoglycerides, Sesame oil, Soybean oil hydrogenated, Capmul MCM (C8/C10 mono-/diglycerides from coconut oil), Benzyl Benzoate, Soybean oil, olive oil, PEG vegetable oil, Vegetable oil, Vegetable oil hydrogenated, peanut oil, mineral oil, or Vegetable shortening.

A “poloxamer” is a block co-polymer. A block refers to a single polyoxyethylene or polyoxypropylene segment. Two-block and three-block polymers have been described. In the case of three-block copolymers, the blocks can be arranged in the format of one polyoxypropylene block surrounded by 2 polyoxyethylene blocks, that being the most common poloxamer structure, or alternatively as one polyoxyethylene block surrounded by 2 polyoxypropylene blocks, the latter sometimes referred to as a reverse poloxamer. Poloxamers are available under the trade names of Lutrol, Monolan, or Pluronic. Poloxamers are also described in U.S. Pat. No. 6,503,955, the complete disclosure of which is hereby incorporated by reference.

“A formulation component” means any substance in addition to the API in a sample. Preferably, a formulation component is therapeutically inactive. Examples of optional formulation components, i.e., other than excipients such as oil, solvent or surfactant, include, but are not limited to, diluents, stabilizers, preservatives, colorants, buffering agents, and combinations thereof.

The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered to a subject in need by injection, e.g., by bolus injection or continuous infusion. Formulations for injection may be presented in unit dosage form, e.g., in ampoules or in multi-dose containers. The term “unit dosage-forms” refers to physically discrete units (e.g., an ampoule) suitable as unitary dosages for human subjects and other mammals, each unit containing a predetermined quantity of lansoprazole calculated to produce the desired therapeutic effect, in association with a suitable excipient system. Formulations for infusion may be presented in forms including drip bags.

Thus, the formulations of the present invention can be provided in forms that possess desired lansoprazole concentrations and are ready for direct administration to a patient. Alternatively, the formulations can be provided in a concentrated form that requires dilution prior to administration. In the case of intravenous administration, the compositions can be admixed with diluents suitable for intravenous administration well known to those experienced in the art. Diluents useful in the formulations of the instant invention may fall within an “excipient category” and include ethanol, propylene glycol, and glycerin. Ethanol is a preferred diluent. Due to the clear and homogenous character of the compositions of the invention, if further diluted, the resulting diluted compositions are generally also homogeneous and clear.

“Compositionally distinct” is used to show that one excipient differs from another excipient in molecular structure, composition, or weight. For example, PEG-300, PEG-400, PEG-540, PEG-600 are “compositionally distinct”.

The term “gastrointestinal disorder” as used herein includes active duodenal ulcers, gastric ulcers, gastroesophageal reflux disease (GERD), severe erosive esophagitis, poorly responsive systematic GERD, and pathological hypersecretory conditions such as Zollinger Ellison syndrome. Dysphagia, reflux oesophagitis, duodenal and benign gastric ulcers, or complications from NSAID therapy, e.g., peri-operative use, are all “gastrointestinal disorders”. The formulations of the instant invention may be administered to a subject in need to treat a gastrointestinal disorder. They may also be administered prophylactically to prevent a gastrointestinal disorder.

“Therapeutically effective amount” when used herein with reference to a formulation of the instant invention denotes a quantity of the formulation which, when administered to a patient or subject, is sufficient to result in a measurable improvement in the state of a gastrointestinal disorder. “Prophylactically effective amount” when used herein with reference to a formulation of the instant invention denotes a quantity of the formulation which, when administered to a patient or subject, is sufficient to prevent a gastrointestinal disorder.

In a preferred embodiment, pharmaceutical formulations of the instant invention may be administered by continuous infusion and comprise about 0.40 mg/mL or more of lansoprazole. Examples of such formulations are provided in Example 1 and Example 2 hereinafter. In another preferred embodiment, pharmaceutical formulations of the instant invention may be administered by injection and comprise about 4.0 mg/ml or more of lansoprazole or an analog, derivative, or salt thereof. Examples of such formulations are provided in Example 3 hereinafter.

The formulations of the present invention preferably have a physiologically neutral pH, such as between about 5 and about 9. The pH of the lansoprazole containing formulations can be adjusted as necessary by, for example, the addition of a base or a salt thereof, for example, an alkali such as sodium hydroxide, potassium hydroxide, or the like. Alternatively, an acid or a salt thereof such as hydrochloric acid, citric acid, or the like can be used to adjust the pH of the compositions.

Pharmaceutical formulations that are intended for application to delicate membranes of the body are commonly adjusted to approximately the same tonicity (i.e., isotonicity) as that of the body fluids. Isotonic compositions are those that cause minimal swelling or contraction of the tissues with which they come in contact, and produce little or no discomfort when instilled in body tissues. Preferably, the lansoprazole formulations of the instant invention are substantially isotonic. The compositions may additionally comprise one or more tonicity modifiers. Examples of tonicity modifiers include, but are not limited to, lactose, dextrose, dextrose anhydrous, mannitol, sodium chloride, potassium chloride, propylene glycol and glycerol.

The lansoprazole formulations are preferably provided or administered as sterile pharmaceutical compositions. For example, the lansoprazole containing formulations are administered substantially free of microorganisms. In some embodiments of the present invention the liquid lansoprazole formulations comprise an antimicrobial, such as disodium edetate, metabisulfate, or a preservatives such as benzyl alcohol, or an antioxidant such as cystein or a salt thereof to retard the growth of microorganisms. In this embodiment, the compositions of the present invention comprise a microbiostatic, microbicidal, preservative, or antioxidant (e.g., cysteine or a salt thereof) in a concentration sufficient to exhibit microbiostatic or microbicidal activity against those microorganisms most likely to contaminate the lansoprazole formulations. A further embodiment includes a sterile pharmaceutical formulation for parenteral administration which comprises lansoprazole and a microbiostatic, microbicidal, preservative, or antioxidant such as cystein (or a salt thereof), EDTA, or metabisulfite, and wherein said aqueous lansoprazole solution is sufficient to prevent no more than a 10-fold increase in growth, or will support no more than a 10-fold increase in growth, of each of Staphylococcus aureus ATCC 6538, Escherichia coli ATCC 8739, Pseudomonas aeruginosa ATCC 9027 and Candida albicans ATCC 10231 for at least 24 hours as measured by a test wherein a washed suspension of each said organism is added to a separate aliquot of said composition at approximately 50 colony forming units per mL, at a temperature in the range 20° C. to 25° C., whereafter said aliquots are incubated at 20° C. to 25° C. for 24 hours and thereafter tested for viable counts of said organism.

The formulations of the invention can optionally include a pharmaceutically acceptable organic acid for stabilization of the formulation during storage and use. These organic acids include, but are not limited to, ascorbic acid, citric acid, tartaric acid, lactic acid, oxalic acid, formic acid, benzene sulphonic acid, benzoic acid, maleic acid, glutamic acid, succinic acid, aspartic acid, diatrizoic acid, or acetic acid. Formulations of the invention preferably include citric acid or a hydrate thereof.

In a further embodiment of the invention, the pharmaceutical formulations further comprise an acid to maintain solution stability, preferably an organic acid such as citric acid or a salt thereof. Citric acid or a salt thereof may exhibit antioxidant and/or chelating properties. Thus, citric acid or a salt thereof may be added to the formulations of the present invention for its favorable effects including but not limited to modifying pH and/or providing or enhancing (a) antioxidant characteristics, (b) chelating effects of the composition, and/or (c) stability of the excipient(s) or the active agent(s). Citric acid or a salt thereof is preferably present in a concentration sufficient to optimize and balance the desired pH and/or the desired antioxidant or chelating properties.

The formulations of the invention can be prepared by combining the API, oils, solvents, surfactants, or other excipients of the invention, and any other components, using well-known pharmaceutical-formulation methods. Formulation of liquid dosage forms is described in Remington: the Science and Practice of Pharmacy, Alfonso R. Gennaro ed., Mack Publishing Co. Easton, Pa., 19th ed., 1995, Chapters 87 and 88; which is incorporated herein by reference. A comprehensive discussion on formulating solutions is presented in Remington: the Science and Practice of Pharmacy, Alfonso R. Gennaro ed., Mack Publishing Co. Easton, Pa., 19th ed., 1995, Chapter 86, which is incorporated herein by reference.

In one process, the formulations of the invention are prepared by dissolving lansoprazole, a derivative or a pharmaceutically acceptable salt thereof in an excipient, e.g., a solvent of the invention, prior to dilution with one or more lansoprazole-free oils, solvents, surfactants, or other excipients as described herein. Such a method increases the amount of lansoprazole that can be formulated and thus delivered parenterally, as well as affecting systemic bioavailability. In another embodiment, the solvent used in this process is a PEG, e.g., PEG-300. In another embodiment, the concentration of lansoprazole after dilution with one or more lansoprazole-free oils, solvents, surfactants or other excipients is greater than 0.3 mg/mL,greater than 0.4 mg/mL, or greater than 40 mg/mL. In still another embodiment, lansoprazole-free oils, solvents, surfactants, and other excipients include, but are not limited to, Triacetin, Transcutol or polysorbate 80. Lansoprazole, as well as all oils, solvents, surfactants, and other excipients of the invention, and any other formulation components, are commercially available or can be synthesized as desired and are preferably purified prior to use in accordance with good manufacturing procedure.

In some embodiments, the excipient or combination of two, three, four, or more than four excipients is present in the composition in a total concentration of about 1 to about 50%, about 2 to 30%, about 2 to 20%, about 2 to 15%, or about 2 to 10% (w/v), for example, about 8%. In another embodiment, the excipient or combination of two, three, four, or more than four excipients is present in the composition in a total concentration less than 40%, less than 30%, less than 29%, less than 28%, less than 27%, less than 26%, less than 25%, less than 24%, less than 23%, less than 22%, less than 21%, less than 20%, less than 19%, less than 18%, less than 17%, less than 16%, less than 15%, less than 14%, less than 13 %, less than 12%, less than 11%, less than 10%, less than 9%, less than 8%, less than 7%, less than 6%, less than 5%, less than 4%, or less than 3% (w/v).

In some embodiments, liquid formulations of the present invention comprise lansoprazole and at least one, at least two, at least three, or at least four excipients.

In one embodiment, lansoprazole is present at a concentration of from about 0.3 to about 50 mg/mL. In another embodiment, lansoprazole is present at a concentration of more than 0.4 mg/mL, more than 1 or 2 mg/mL, more than 3 mg/mL, more than 4 mg/mL, more than 5 mg/mL, more than 6 mg/mL, more than 7 mg/mL, more than 8 nig/mL, more than 9 mg/mL, more than 10 mg/mL, more than 11 mg/mL, more than 12 mg/mL, more than 13 mg/mL, more than 14 mg/mL, more than 15 mg/mL, more than 16 mg/mL, more than 17 mg/mL, more than 18 mg/mL, more than 19 mg/mL, more than 20 mg/mL, more than 21 mg/mL, more than 22 mg/mL, more than 23 mg/mL, more than 24 mg/mL, more than 25 mg/mL, more than 26 mg/mL, more than 27 mg/mL, more than 28 mg/mL, more than 29 mg/mL, more than 30 mg/mL, more than 31 mg/mL, more than 31 mg/mL, more than 32 mg/mL, more than 33 mg/mL, more than 34 mg/mL, more than 35 mg/mL, more than 36 mg/mL, more than 37 mg/mL, more than 38 mg/mL, more than 39 mg/mL, more than 40 mg/mL, more than 41 mg/mL, more than 42 mg/mL, more than 43 mg/mL, more than 44 mg/mL, more than 45 mg/mL, more than 46 mg/mL, more than 47 mg/mL, more than 48 mg/mL, more than 49 mg/mL, and about 50 mg/mL. Alternatively, between about 0.3 and about 40, about 0.4 and about 20, or about 0.4 and about 4 mg of lansoprazole per mL of composition are present. Preferably, lansoprazole is present at about 0.4 to about 4 milligrams per milliliter of composition.

Alternatively, lansoprazole compositions can be expressed as lansoprazole percent weight/volume (w/v). For example, compositions of the invention can have lansoprazole compositions of at least 0.5, 0.75, 1.0, 1.25, 1.5, 1.75, 2.0, 2.25, 2.5, 2.75, 3.0, 4, 5, 6, 7, 8, 9, 10, 11, 12, 13, 14, 15, 16, 17, 18, 19, 20, 21, 22, 23, 24, or 25 percent (w/v), or 0.5 to about 2.4, about 0.5 to about 2, about 0.5 to about 1.5, about 0.8 to about 1.2, or about 0.9 to about 1.1 percent (w/v).

In another embodiment of the present invention, the ratio of excipients in a liquid formulation can be altered to change the concentration of lansoprazole. For example, in a two excipient system, the ratio of excipients can be 1:1, 1:0.5, 3:1, 4:1, etc. Similarly, in a three excipient system, the ratio of excipients can be, for example, 1:1:1, 2:1:1, 1:2:3, 1:0.5:0.1, etc. Four excipient systems and higher order systems can likewise be prepared in many distinct ratios of excipients.

Formulations of the instant invention that are to be administered by continuous infusion preferably have lansoprazole concentrations of about 0.3 mg/mL or 0.4 mg/mL or greater. (All concentrations referred to herein are based on the total volume of the formulation.) Formulations of the instant invention that are to be administered by bolus injection preferably have lansoprazole concentrations of about 4 mg/mL or greater.

The exact amount of lansoprazole that may be combined with the excipient system to produce a single dosage form will vary depending upon the subject treated and the particular parenteral mode of administration. For example, a dosage regimen of about 40 mg per day of lansoprazole administered for a period of about five days may be required to treat a human suffering from dysphagia, reflux oesophagitis, duodenal and benign gastric ulcers, or complications from NSAID therapy. For administration of such a dosage regimen by intravenous injection to a human subject, a once-a-day injection of a 10 mL bolus comprising an injectable formulation of the instant invention having a lansoprazole concentration of about 4 mg/mL can be used. Such an injection should take about five minutes on average to administer. For administration of such a dosage regimen by continuous infusion to a human subject, a once-a-day infusion of a 100 mL infusion formulation of the instant invention having a lansoprazole concentration of about 0.4 mg/mL can be used. Such an infusion should take about twenty to thirty minutes on average to administer.

In another embodiment of the present invention, a liquid lansoprazole formulation comprises a one or a two excipient system selected from the group consisting of: lecithin; lecithin and polyvinylpyrrolidone; lecithin and sorbitol; lecithin and lysine; lecithin and PEG 12; lecithin and PEG 400; lecithin and poloxamer 188; lecithin and polysorbate 80; lecithin and polysorbate 20; methylparaben and sorbitol; methylparaben and polysorbate 80; methylparaben and polysorbate 20; gamma-cyclodextrin and lecithin; gamma-cyclodextrin and polyvinylpyrrolidone; gamma-cyclodextrin and sorbitol; gamma-cyclodextrin and sodium acetate; gamma-cyclodextrin and sodium benzoate; gamma-cyclodextrin and poloxamer 188; gamma-cyclodextrin and polysorbate 80; gamma-cyclodextrin and propylene glycol; gamma-cyclodextrin and polysorbate 20; calcium gluceptate and sorbitol; calcium gluceptate and diethanolamine; calcium gluceptate and PEG 35 castor oil; calcium gluceptate and poloxamer 188; calcium gluceptate and polysorbate 80; calcium gluceptate and polysorbate 20; deoxycholic acid and lecithin; deoxycholic acid and methylparaben; deoxycholic acid and gamma-cyclodextrin; deoxycholic acid; deoxycholic acid and mannitol; deoxycholic acid and polyvinylpyrrolidone; deoxycholic acid and sorbitol; deoxycholic acid and diethanolamine; deoxycholic acid and lysine; deoxycholic acid and magnesium chloride; deoxycholic acid and PEG 12; deoxycholic acid and sodium acetate; deoxycholic acid and sodium benzoate; deoxycholic acid and sodium tartrate; deoxycholic acid and ethanol; deoxycholic acid and glycerin; deoxycholic acid and hydroxypropyl-beta-cyclodextrin; deoxycholic acid and PEG 400; deoxycholic acid and PEG 6; deoxycholic acid and poloxamer 188; deoxycholic acid and polysorbate 80; deoxycholic acid and propylene glycol; deoxycholic acid and polysorbate 20; lactose and deoxycholic acid; lactose and polyvinylpyrrolidone; lactose and sorbitol; lactose and benzethonium chloride; lactose and diethanolamine; lactose and PEG 35 castor oil; lactose and poloxamer 188; lactose and polysorbate 80; lactose and polysorbate 20; mannitol and sorbitol; mannitol and poloxamer 188; mannitol and polysorbate 80; mannitol and polysorbate 20; polyvinylpyrrolidone; polyvinylpyrrolidone and sorbitol; polyvinylpyrrolidone and sodium benzoate; polyvinylpyrrolidone and sodium tartrate; polyvinylpyrrolidone and polysorbate 80; polyvinylpyrrolidone and polysorbate 20; sorbitol; sorbitol and polysorbate 80; sorbitol and polysorbate 20; chlorobutanol and sorbitol; chlorobutanol and PEG 35 castor oil; chlorobutanol and polysorbate 80; benzethonium chloride and calcium gluceptate; benzethonium chloride and chlorobutanol; benzethonium chloride; benzethonium chloride and PEG 35 castor oil; benzethonium chloride and polysorbate 80; diethanolamnine and lecithin; diethanolamine and gamma-cyclodextrin; diethanolamine and mannitol; diethanolamine and polyvinylpyrrolidone; diethanolamine and sorbitol; diethanolamine; diethanolamine and lysine; diethanolamine and sodium acetate; diethanolamine and ethanol; diethanolamine and glycerin; diethanolamine and hydroxypropyl-beta-cyclodextrin; diethanolamine and PEG 400; diethanolamine and PEG 6; diethanolamine and poloxamer 188; diethanolamine and polysorbate 80; diethanolamine and propylene glycol; diethanolamine and polysorbate 20; lysine and polyvinylpyrrolidone; lysine and sorbitol; lysine and poloxamer 188; lysine and polysorbate 80; lysine and polysorbate 20; magnesium chloride and sorbitol; magnesium chloride and poloxamer 188; magnesium chloride and polysorbate 80; magnesium chloride and polysorbate 20; PEG 12 and polyvinylpyrrolidone; PEG 12 and sorbitol; PEG 12 and poloxamer 188; PEG 12 and polysorbate 80; PEG 12 and polysorbate 20; sodium acetate and sorbitol; sodium acetate and polysorbate 80; sodium acetate and polysorbate 20; sodium benzoate and sorbitol; sodium benzoate; sodium benzoate and polysorbate 80; sodium benzoate and polysorbate 20; sodium tartrate and sorbitol; sodium tartrate and polysorbate, 80; sodium tartrate and polysorbate 20; ethanol and sorbitol; ethanol and glycerin; ethanol and hydroxypropyl-beta-cyclodextrin; ethanol and poloxamer 188; ethanol and polysorbate 80; ethanol and propylene glycol; ethanol and polysorbate 20; glycerin and lecithin; glycerin and polyvinylpyrrolidone; glycerin and sorbitol; glycerin and hydroxypropyl-beta-cyclodextrin; glycerin and poloxamer 188; glycerin and polysorbate 80; glycerin and polysorbate 20; hydroxypropyl-beta-cyclodextrin and methylparaben; hydroxypropyl-beta-cyclodextrin and mannitol; hydroxypropyl-beta-cyclodextrin and polyvinylpyrrolidone; hydroxypropyl-beta-cyclodextrin and sorbitol; hydroxypropyl-beta-cyclodextrin and PEG 12; hydroxypropyl-beta-cyclodextrin and sodium acetate; hydroxypropyl-beta-cyclodextrin and sodium benzoate; hydroxypropyl-beta-cyclodextrin and sodium tartrate; hydroxypropyl-beta-cyclodextrin and poloxamer 188; hydroxypropyl-beta-cyclodextrin and polysorbate 80; hydroxypropyl-beta-cyclodextrin and polysorbate 20; PEG 35 castor oil and lecithin; PEG 35 castor oil and methylparaben; PEG 35 castor oil and gamma-cyclodextrin; PEG 35 castor oil and deoxycholic acid; PEG 35 castor oil and mannitol; PEG 35 castor oil and polyvinylpyrrolidone; PEG 35 castor oil and sorbitol; PEG 35 castor oil and diethanolamine; PEG 35 castor oil and lysine; PEG 35 castor oil and magnesium chloride; PEG 35 castor oil and PEG 12; PEG 35 castor oil and sodium acetate; PEG 35 castor oil and sodium benzoate; PEG 35 castor oil and sodium tartrate; PEG 35 castor oil and ethanol; PEG 35 castor oil and glycerin; PEG 35 castor oil and hydroxypropyl-beta-cyclodextrin; PEG 35 castor oil and PEG 400; PEG 35 castor oil and PEG 6; PEG 35 castor oil and poloxamer 188; PEG 35 castor oil and polysorbate 80; PEG 35 castor oil and propylene glycol; PEG 35 castor oil and polysorbate 20; PEG 400 and sorbitol; PEG 400 and poloxamer 188; PEG 400 and polysorbate 80; PEG 400 and polysorbate 20; PEG 6 and sorbitol; PEG 6 and poloxamer 188; PEG 6 and polysorbate 80; PEG 6 and polysorbate 20; poloxamer 188 and polyvinylpyrrolidone; poloxamer 188 and sorbitol; poloxamer 188 and sodium acetate; poloxamer 188 and sodium benzoate; poloxamer 188 and sodium tartrate; poloxamer 188; poloxamer 188 and polysorbate 80; poloxamer 188 and propylene glycol; poloxamer 188 and polysorbate 20; polysorbate 80; propylene glycol and sorbitol; propylene glycol and polysorbate 80; propylene glycol; propylene glycol and polysorbate 20; polysorbate 20 and polysorbate 80; polysorbate 20; lactose and methylparaben; mannitol and polyvinylpyrrolidone; mannitol and sodium acetate; polyvinylpyrrolidone and sodium acetate; chlorobutanol and polysorbate 20; benzethonium chloride and lecithin; benzethonium chloride and methylparaben; benzethonium chloride and gamma-cyclodextrin; benzethonium chloride and mannitol; benzethonium chloride and polyvinylpyrtolidone; benzethonium chloride and sorbitol; benzethonium chloride and diethanolamine; benzethonium chloride and lysine; benzethonium chloride and PEG 12; benzethonium chloride and sodium acetate; benzethonium chloride and sodium tartrate; benzethonium chloride and ethanol; benzethonium chloride and glycerin; benzethonium chloride and hydroxypropyl-beta-cyclodextrin; benzethonium chloride and PEG 400; benzethonium chloride and PEG 6; benzethonium chloride and poloxamer 188; benzethonium chloride and propylene glycol; benzethonium chloride and polysorbate 20; diethanolamine and methylparaben; diethanolamine and magnesium chloride; diethanolamine and PEG 12; diethanolamine and sodium benzoate; diethanolamine and sodium tartrate; magnesium chloride and polyvinylpyrrolidone; PEG 12 and sodium acetate; ethanol and lecithin; ethanol and gamma-cyclodextrin; ethanol and polyvinylpyrrolidone; ethanol and sodium acetate; hydroxypropyl-beta-cyclodextrin and lysine; hydroxypropyl-beta-cyclodextrin and magnesium chloride; hydroxypropyl-beta-cyclodextrin and PEG 400; hydroxypropyl-beta-cyclodextrin and PEG 6; hydroxypropyl-beta-cyclodextrin and propylene glycol; propylene glycol and polyvinylpyrrolidone; lecithin and methylparaben; lecithin and mannitol; lecithin and sodium acetate; lecithin and sodium benzoate; lecithin and sodium tartrate; lecithin and PEG 6; lecithin and propylene glycol; methylparaben and PEG 12; methylparaben and PEG 6; methylparaben and poloxamer 188; gamma-cyclodextrin and methylparaben; gamma-cyclodextrin and PEG 12; calcium gluceptate and ethanol; lactose and lecithin; lactose and lysine; lactose and sodium benzoate; lactose and sodium tartrate; lactose and glycerin; lactose and PEG 6; mannitol and methylparaben; mannitol and PEG 400; mannitol and propylene glycol; chlorobutanol and lecithin; chlorobutanol and diethanolamine; benzethonium chloride and sodium benzoate; lysine and methylparaben; lysine and mannitol; lysine; lysine and PEG 6; magnesium chloride; magnesium chloride and sodium acetate; magnesium chloride and PEG 400; sodium tartrate; ethanol and methylparaben; ethanol and mannitol; ethanol and sodium benzoate; ethanol and PEG 400; ethanol and PEG 6; hydroxypropyl-beta-cyclodextrin and lecithin; PEG 35 castor oil; and propylene glycol and sodium acetate.

Other embodiments of the present invention specifically include, but are not limited to, two, three, and four excipient systems such as: polysorbate 80 and PEG 400; polysorbate 80 and polypropylene glycol; polysorbate 80 and ethanol; PEG 300 and polypropylene glycol; polysorbate 20 and PEG 300; a 3:1 volumetric ratio of polysorbate 80 and polypropylene glycol; a 0.3:0.8:0.2 volumetric ratio of polysorbate 80, polypropylene glycol, and ethanol; a 2.5:1.0:0.5 volumetric ratio of polysorbate 80, polypropylene glycol, and PEG 300; and a 2:1:0.8:0.2 volumetric ratio of polysorbate 80, polypropylene glycol, PEG 300, and ethanol. Any combination of any two, three, four or more of the excipients listed herein may be combined into an excipient system for lansoprazole. Similarly, any relative ratio of excipients in an excipient system may be used in accordance with the present invention.

Included as embodiments of the present invention are compositions or formulations that exclude a specified excipient. Any known excipient, including those disclosed herein or disclosed in Handbook of Pharmaceutical Additives compiled by Michael and Irene Ash, Gower Publishing, 1995 (incorporated herein by reference in its entirety), may be specifically excluded from the present invention. Any one or more than one species of excipients may be excluded from the present invention. For e.g., D-alpha-tocopheryl polyethylene glycol 1000 succinate may be excluded from the present invention. Compositions or formulations that comprise a specific excipient exceeding a specified amount may also be excluded. For example, a composition or formulation comprising a specified excipient(s) with a concentration of 90% or more, 80% or more, 70% or more, 60% or more, 50% or more, 40% or more, 30% or more, 29% or more, 28% or more, 27% or more, 26% or more, 25% or more, 24% or more, 23% or more, 22% or more, 21% or more, 20% or more, 19% or more, 18% or more, 17% or more, 16%ormore, 15%ormore, 14%ormore, 13%ormore, 12%ormore, 11% or more, 10% or more, 9% or more, 8% or more, 7% or more, 6% or more, 5% or more, 4% or more, 3% or more, 2% or more,or 1% or more (w/v) may be specifically excluded from the present invention. For example, the following may be specifically excluded from the present invention: 8% or more, or 10% or more of D-alpha-tocopheryl polyethylene glycol 1000 succinate (w/v); 10% or more or 20% or more of hydroxypropyl-beta-cyclodextrin (w/v); 5% or more, or 30% or more of N-methylpyrrolidone or pyrrolidone, 30% or more of propylene glycol (w/v); combination of either N-methylpyrrolidone or pyrrolidone, and propylene glycol (or a combination of all three), wherein the combined concentration is 60% or more (w/v); 2.5% or more, or 5% or more of a bile acid salt (e.g., sodium glycocolate/glycocolic acid), 4% or more, or 7% or more of lecithin (e.g., soybean or egg), or a combined concentration of 5% or more, or 7.5% or more, or 10% or more of both a bile salt and a lecithin (w/v); 0.5% or more, or 1% or more of benzyl alcohol (w/v); 5% or more, or 15% or more of polyethoxylated castor oil (w/v); 5% or more, 7.5% or more, or 10% or more of a cyclodextrin, such as a sulfoalkyl ether cyclodextrin or sulfobutyl ether cyclodextrin. Categories of excipients may also be specifically included or excluded as a component of a composition or formulation of the present invention, and optionally including the concentrations.

As a further embodiment, compositions or formulations of the present invention may be limited to compositions or formulations comprising excipients consisting of excipients; a) certified as GRAS [Generally Recognized as Safe] by the Food and Drug Administration (FDA), b) approved as a food additive pursuant to 21 CFR 171, or c) approved by the FDA for a specific application through a new drug application, and optionally at or below the concentration approved.

It should also be understood that a specific dosage and treatment regimen for any particular patient will depend upon a variety of factors, including age, body weight, general health, sex, diet, time of administration, rate of excretion, drug combination, and the judgment of the treating physician and the severity of the particular disease or injury being treated. Furthermore, discussion of the parenteral administration of the lansoprazole formulations of the present invention is not intended to limit the invention. In another embodiment, a lansoprazole formulation of the present invention can be used for oral administration. Such an oral dosage form can be used to treat a subject in need of therapeutic treatment.

According to an alternate embodiment, the invention provides a method of administering lansoprazole in a single or multiple dosages. If separate dosages are utilized, they may be administered concurrently or consecutively.

The invention is described further in the following examples, which are illustrative and in no way limiting.

### EXAMPLE 1

Formulations for Continuous Infusion

A variety of lansoprazole one and two-excipient system formulations were made, based on permutations of types and amounts of the excipients listed in Table 1. Approximately 100 microliter samples of each excipient were used. These formulations were subjected to sequential dilution testing using the FAST® integrated series of high-throughput, automated instrumentation to determine lansoprazole concentration. The methods and systems referred to as FAST® are described in U.S. patent application Ser. No. 09/628,667, the entirety of which is incorporated herein by reference. The meaurement of concentration was completed via UV spectroscopy. Those formulations in which lansoprazole concentration was determined to be greater than about 0.4 mg/mL or greater were identified as preferred formulations for administration by continuous infusion. 275 formulations that used eleven categories of lansoprazole one and two-excipient systems were determined to have a lansoprazole concentration of about 0.4 mg/mL or greater. These preferred formulations are listed in Table 2. Nearly all lansoprazole concentrations of these formulations ranged from about 0.4 mg/mL to about 4.0 mg/mL.

In Table 2, “RSD (API)” is the relative standard deviation of lansoprazole concentrations determined during testing. Additionally, in Table 2, pre-dilution excipient concentrations are listed before the name of each excipient and lansoprazole concentrations are listed under the “API Conc. (mg/mL)” column.

TABLE 1
Aqueous Excipient List

| All excipients are in 0.1M pH | | | | Concentration of | | pH with | UV |
|---|---|---|---|---|---|---|---|
| 8.790 Tris Buffer | | | Volume | Excipient with Tris | | Tris | Absorbance |
| | Excipient | Mass (g) | (ml) | Buffer (mg/ml) | pH | Buffer | at λ = 310 nm |
| 1 | α Lactose | 5.04 | 100 | 25 | 4.6 | 8.8 | 0.058 |
| 2 | Benzethonium Chloride | 8.00 | 100 | 40 | 6.2 | 8.8 | 0.070 |
| 3 | calcium gluceptate | 8.01 | 200 | 20 | 7.4 | 8.8 | 0.055 |
| 4 | chlorobutanol | 0.801 | 125 | 3.2 | 5.0 | 8.8 | 0.054 |
| 5 | Cremophor EL | 10.0 | 100 | 50 | 5.8 | 8.7 | 0.212 |
| 6 | Deoxycholic Acid | 5.07 | 100 | 25 | 8.6 | 8.9 | 0.060 |
| 7 | Diethanolamine | 8.00 | 100 | 40 | 11.5 | 10.9 | 0.057 |
| 8 | Ethanol | 10.0 | 100 | 50 | 6.5 | 8.7 | 0.054 |
| 9 | Gamma Cyclodextrin | 2.80 | 100 | 14 | 5.2 | 8.8 | 0.057 |
| 10 | glycerin | 10.0 | 100 | 50 | 5.0 | 8.8 | 0.049 |
| 11 | hydroxypropyl-beta- | 10.1 | 100 | 50 | 7.2 | 8.8 | 0.067 |
| | cyclodextrin | | | | | | |
| 12 | lecithin | 10.0 | 7813 | 0.64 | 5.7 | 8.8 | 0.267 |
| 13 | lysine | 8.02 | 100 | 40 | 5.6 | 7.9 | 0.058 |
| 14 | magnesium chloride | 8.02 | 100 | 40 | 6.7 | 8.6 | 0.049 |
| 15 | Mannitol | 5.06 | 100 | 25 | 5.9 | 8.8 | 0.049 |
| 16 | Methylparaben | N/A | N/A | N/A | N/A | N/A | 3.120 |
| 17 | PEG 300 | 10.0 | 100 | 50 | 5.2 | 8.8 | 0.048 |
| 18 | PEG 400 | 10.0 | 100 | 50 | 4.4 | 8.7 | 0.050 |
| 19 | PEG 600 | 8.01 | 100 | 40 | 3.6 | 8.7 | 0.047 |
| 20 | poloxamer 188 | 10.1 | 100 | 50 | 7.2 | 8.8 | 0.048 |
| 21 | Propylene glycol | 10.1 | 100 | 50 | 5.5 | 8.8 | 0.053 |
| 22 | Polyvinylpyrrolidone | 5.04 | 100 | 25 | 4.3 | 8.5 | 0.049 |
| 23 | Sodium acetate | 8.01 | 100 | 40 | 8.6 | 8.9 | 0.224 |
| 24 | Sodium benzoate | 8.01 | 100 | 40 | 7.0 | 8.9 | 0.047 |
| 25 | Sodium tartrate | 8.05 | 100 | 40 | 7.7 | 9.0 | 0.056 |
| 26 | Sorbitol | 5.06 | 100 | 25 | 4.5 | 8.8 | 0.051 |
| 27 | Polysorbate 20 | 10.0 | 1000 | 5.0 | 5.5 | 8.7 | 0.086 |
| 28 | Polysorbate 80 | 10.0 | 100 | 50 | 6.1 | 8.6 | 0.199 |

In the case of the listed single excipient systems (e.g., formulation 1 of Table 2), the excipient concentration value in the diluted formulation is the same as that listed in Table 2. Where two different excipients were used, the excipient concentrations in the diluted formulations were one-half of those listed in Table 2. Using formulations 1 and 2 of Table 2 as examples, the lecithin concentration in formulation 1 was 0.6 mg/mL (adding two lecithin 100 μl samples which each had a 0.6 mg/mL concentration resulted in a final lecithin concentration of 0.6 mg/mL). In formulation 2 of Table 2, the lecithin concentration in the diluted formulations was 0.3 mg/mL and the polyvinylpyrrolidone concentration in the diluted formulations was 12.5 mg/mL.

Generally, the preferred lansoprazole one and two-excipient systems are identified in Table 2. These comprised lansoprazole and a single-excipient, or lansoprazole in combination with a two-excipient system comprising either compositionally distinct first and second excipients selected from the same excipient category, or compositionally distinct first and second excipients selected from different excipient categories. The eleven excipient categories were as follows: hydrotropes, preservatives, pharmaceutically acceptable salts, surfactants, bases, cyclodextrins, viscosity modifiers, emulsifiers, solvents, carriers, and lubricants. Total excipient concentrations in these one and two-excipient systems ranged from about 0.6 mg/mL to about 50 mg/mL. All of the formulations were made at about room temperature and atmospheric pressure. These preferred eleven categories of lansoprazole one and two-excipient systems were categorized as follows.

Lansoprazole Infusion Formulations Using One and Two-Excipient Systems

1. A single-excipient system comprising an emulsifier, or a two-excipient system comprising either (1) compositionally distinct first and second emulsifiers, or (2) an emulsifier in combination with one of the following: (i) a viscosity modifier, (ii) a carrier, (iii) a base, (iv) a solvent, or (v) a surfactant. These are identified, for example, in entries 1-9 of Table 2.

2. A two-excipient system comprising a preservative in combination with one of the following: (i) a carrier, (ii) a surfactant, (iii) a solvent, or (iv) a cyclodextrin. These are identified, for example, in entries 10-12, 29, and 148 of Table 2.

3. A single-excipient system comprising a cyclodextrin, or a two-excipient system comprising either (1) compositionally distinct first and second cyclodextrins, or (2) a cyclodextrin in combination with one of the following: (i) an emulsifier, (ii) a viscosity modifier, (iii) a carrier, (iv) a lubricant, (v) a surfactant, or (vi).a solvent. These are identified, for example, in entries 13-21,124, and 136-144 of Table 2.

4. A two-excipient system comprising calcium salt in combination with one of the following: (i) a carrier, (ii) a base, (iii) a solvent, or (iv) a surfactant. These are identified, for example, in entries 22-27 of Table 2.

5. A single-excipient system comprising a surfactant, or a two-excipient system comprising either (1) compositionally distinct first and second surfactants, or (2) a surfactant in combination with one of the following: (i) a carrier, (ii) a viscosity modifier, (iii) a base, (iv) a pharmaceutically acceptable salt that is not a calcium salt, (v) a solvent, (vi) a lubricant, or (vii) a hydrotrope. These are identified, for example, in entries 31-50 of Table 2.

6. A two-excipient system comprising a hydrotrope in combination with one of the following: (i) a viscosity modifier, (ii) a carrier, (iii) a preservative, (iv) a base, (v) a solvent, or (vi) a cyclodextrin. These are identified, for example, in entries 52-56, 123, and 132 of Table 2.

7. A single-excipient system comprising a viscosity modifier, or a two-excipient system comprising a viscosity modifier in combination with one of the following: (i) a carrier, (ii) a lubricant, or (iii) a solvent. These are identified, for example, in entries 64-67 and 152 of Table 2.

8. A single-excipient system comprising a carrier, or a two-excipient system comprising a carrier in combination with one of the following: (i) a solvent or (ii) a lubricant. These are identified, for example, in entries 70, 108, 115, and 122 of Table 2.

9. A single-excipient system comprising a base, or a two-excipient system comprising either (1) compositionally distinct first and second bases, or (2) a base in combination with one of the following: (i) a preservative, (ii) a solvent, (iii) a carrier, (iv) a viscosity modifier, or (vi) a cyclodextrin. These are identified, for example, in entries 82-89 and 91-93 of Table 2.

10. A single-excipient system comprising a solvent, or a two-excipient system comprising either (1) compositionally distinct first and second solvents, or (2) a solvent in combination with one of the following: (i) a salt or (ii) a lubricant. These are identified, for example, in entries 156-161, and 190 of Table 2.

11. A single-excipient system comprising a lubricant. A single lubricant system is identified in entry 116 of Table 2.

Infusion Formulation Dosage Forms

The preferred lansoprazole formulations of Table 2 are suitable for administration by infusion. The excipient concentrations for the formulations (determined as described above) and the lansoprazole concentrations are working concentrations, i.e., concentrations of the formulation as administered to the patient. Lansoprazole stock solutions may be prepared, e.g., for storage in a vial, as either lansoprazole plus pure excipient or in a formulation diluted to a desired concentration by an aqueous solvent. Formulations for infusion preferably will have a lansoprazole concentration of about 0.4 mg/mL. A preservative may be added to the formulations and the formulations can be ultimately administered to a patient from drip bags or other known dosage systems.

TABLE 2

| | | | API Conc. | |
|---|---|---|---|---|
| | Excipient 1 | Excipient 2 | (mg/mL) | RSD (API) |
| 1 | 0.6 mg/mL Lecithin | 0.6 mg/mL Lecithin | 0.51 | 0.041 |
| 2 | 0.6 mg/mL Lecithin | 25 mg/mL | 0.81 | 0.039 |
| | | Polyvinylpyrrolidone | | |
| 3 | 0.6 mg/mL Lecithin | 25 mg/mL Sorbitol | 1.43 | 0.018 |
| 4 | 0.6 mg/mL Lecithin | 40 mg/mL Lysine | 0.68 | 0.022 |
| 5 | 0.6 mg/mL Lecithin | 40 mg/mL PEG-12 | 0.84 | 0.04 |
| 6 | 0.6 mg/mL Lecithin | 50 mg/mL PEG-400 | 0.73 | 0.068 |
| 7 | 0.6 mg/mL Lecithin | 50 mg/mL Poloxamer 188 | 0.81 | 0.019 |
| 8 | 0.6 mg/mL Lecithin | 50 mg/mL Polysorbate 80 | 1.91 | 0.022 |
| 9 | 0.6 mg/mL Lecithin | 5 mg/mL Polysorbate 20 | 2.21 | 0.03 |
| 10 | 1.0 mg/mL Methylparaben | 25 mg/mL Sorbitol | 2.17 | 0.022 |
| 11 | 1.0 mg/mL Methylparaben | 50 mg/mL Polysorbate 80 | 1.76 | 0.02 |
| 12 | 1.0 mg/mL Methylparaben | 5 mg/mL Polysorbate 20 | 2.65 | 0.045 |
| 13 | 14 mg/mL Gamma- | 0.6 mg/mL Lecithin | 1.07 | 0.027 |
| | Cyclodextrin | | | |
| 14 | 14 mg/mL Gamma- | 25 mg/mL | 0.64 | 0.024 |
| | Cyclodextrin | Polyvinylpyrrolidone | | |
| 15 | 14 mg/mL Gamma- | 25 mg/mL Sorbitol | 2.15 | 0.039 |
| | Cyclodextrin | | | |
| 16 | 14 mg/mL Gamma- | 40 mg/mL Sodium Acetate | 0.75 | 0.053 |
| | Cyclodextrin | | | |
| 17 | 14 mg/mL Gamma- | 40 mg/mL Sodium benzoate | 0.43 | 0.037 |
| | Cyclodextrin | | | |
| 18 | 14 mg/mL Gamma- | 50 mg/mL Poloxamer 188 | 1.33 | 0.033 |
| | Cyclodextrin | | | |
| 19 | 14 mg/mL Gamma- | 50 mg/mL Polysorbate 80 | 1.73 | 0.02 |
| | Cyclodextrin | | | |
| 20 | 14 mg/mL Gamma- | 50 mg/mL Propylene glycol | 1.13 | 0.016 |
| | Cyclodextrin | | | |
| 21 | 14 mg/mL Gamma- | 5 mg/mL Polysorbate 20 | 2.31 | 0.023 |
| | Cyclodextrin | | | |
| 22 | 20 mg/mL Calcium gluceptate | 25 mg/mL Sorbitol | 1.39 | 0.034 |
| 23 | 20 mg/mL Calcium gluceptate | 40 mg/mL Diethanolamine | 1.05 | 0.028 |
| 24 | 20 mg/mL Calcium gluceptate | 50 mg/mL PEG-35 castor oil | 1.91 | 0.012 |
| 25 | 20 mg/mL Calcium gluceptate | 50 mg/mL Poloxamer 188 | 0.84 | 0.044 |
| 26 | 20 mg/mL Calcium gluceptate | 50 mg/mL Polysorbate 80 | 1.31 | 0.069 |
| 27 | 20 mg/mL Calcium gluceptate | 5 mg/mL Polysorbate 20 | 0.94 | 0.01 |
| 28 | 25 mg/mL Deoxycholic Acid | 0.6 mg/mL Lecithin | 1.62 | 0.036 |
| 29 | 25 mg/mL Deoxycholic Acid | 1.0 mg/mL Methylparaben | 1.79 | 0.041 |
| 30 | 25 mg/mL Deoxycholic Acid | 14 mg/mL Gamma- | 1.80 | 0.034 |
| | | Cyclodextrin | | |
| 31 | 25 mg/mL Deoxycholic Acid | 25 mg/mL Deoxycholic Acid | 1.54 | 0.029 |
| 32 | 25 mg/mL Deoxycholic Acid | 25 mg/mL Mannitol | 1.39 | 0.027 |
| 33 | 25 mg/mL Deoxycholic Acid | 25 mg/mL | 1.67 | 0.014 |
| | | Polyvinylpyrrolidone | | |
| 34 | 25 mg/mL Deoxycholic Acid | 25 mg/mL Sorbitol | 2.34 | 0.01 |
| 35 | 25 mg/mL Deoxycholic Acid | 40 mg/mL Diethanolamine | 1.56 | 0.064 |
| 36 | 25 mg/mL Deoxycholic Acid | 40 mg/mL Lysine | 1.33 | 0.028 |
| 37 | 25 mg/mL Deoxycholic Acid | 40 mg/mL Magnesium | 1.64 | 0.027 |
| | | chloride | | |
| 38 | 25 mg/mL Deoxycholic Acid | 40 mg/mL PEG-12 | 1.52 | 0.024 |
| 39 | 25 mg/mL Deoxycholic Acid | 40 mg/mL Sodium Acetate | 1.56 | 0.02 |
| 40 | 25 mg/mL Deoxycholic Acid | 40 mg/mL Sodium benzoate | 1.64 | 0.022 |
| 41 | 25 mg/mL Deoxycholic Acid | 40 mg/mL Sodium tartrate | 1.62 | 0.036 |
| 42 | 25 mg/mL Deoxycholic Acid | 50 mg/mL Ethanol | 1.46 | 0.043 |
| 43 | 25 mg/mL Deoxycholic Acid | 50 mg/mL Glycerin | 1.29 | 0.028 |
| 44 | 25 mg/mL Deoxycholic Acid | 50 mg/mL Hydroxypropyl-b- | 1.54 | 0.016 |
| | | cyclodextrin | | |
| 45 | 25 mg/mL Deoxycholic Acid | 50 mg/mL PEG-400 | 1.47 | 0.029 |
| 46 | 25 mg/mL Deoxycholic Acid | 50 mg/mL PEG-6 | 1.44 | 0.017 |
| 47 | 25 mg/mL Deoxycholic Acid | 50 mg/mL Poloxamer 188 | 1.81 | 0.042 |
| 48 | 25 mg/mL Deoxycholic Acid | 50 mg/mL Polysorbate 80 | 2.33 | 0.012 |
| 49 | 25 mg/mL Deoxycholic Acid | 50 mg/mL Propylene glycol | 1.73 | 0.012 |
| 50 | 25 mg/mL Deoxycholic Acid | 5 mg/mL Polysorbate 20 | 3.18 | 0.047 |
| 51 | 25 mg/mL Lactose | 25 mg/mL Deoxycholic Acid | 1.87 | 0.015 |
| 52 | 25 mg/mL Lactose | 25 mg/mL | 0.44 | 0.062 |
| | | Polyvinylpyrrolidone | | |
| 53 | 25 mg/mL Lactose | 25 mg/mL Sorbitol | 1.78 | 0.033 |
| 54 | 25 mg/mL Lactose | 40 mg/mL Benzethonium | 0.86 | 0.024 |
| | | chloride | | |
| 55 | 25 mg/mL Lactose | 40 mg/mL Diethanolamine | 1.03 | 0.079 |
| 56 | 25 mg/mL Lactose | 50 mg/mL PEG-35 castor oil | 2.59 | 0.055 |
| 57 | 25 mg/mL Lactose | 50 mg/mL Poloxamer 188 | 1.07 | 0.01 |
| 58 | 25 mg/mL Lactose | 50 mg/mL Polysorbate 80 | 1.91 | 0.026 |
| 59 | 25 mg/mL Lactose | 5 mg/mL Polysorbate 20 | 1.75 | 0.026 |
| 60 | 25 mg/mL Mannitol | 25 mg/mL Sorbitol | 1.81 | 0.044 |
| 61 | 25 mg/mL Mannitol | 50 mg/mL Poloxamer 188 | 1.33 | 0.09 |
| 62 | 25 mg/mL Mannitol | 50 mg/mL Polysorbate 80 | 1.60 | 0.022 |
| 63 | 25 mg/mL Mannitol | 5 mg/mL Polysorbate 20 | 1.93 | 0.015 |
| 64 | 25 mg/mL | 25 mg/mL | 0.41 | 0.048 |
| | Polyvinylpyrrolidone | Polyvinylpyrrolidone | | |
| 65 | 25 mg/mL | 25 mg/mL Sorbitol | 1.47 | 0.044 |
| | Polyvinylpyrrolidone | | | |
| 66 | 25 mg/mL | 40 mg/mL Sodium benzoate | 0.40 | 0.049 |
| | Polyvinylpyrrolidone | | | |
| 67 | 25 mg/mL | 40 mg/mL Sodium tartrate | 0.88 | 0.075 |
| | Polyvinylpyrrolidone | | | |
| 68 | 25 mg/mL | 50 mg/mL Polysorbate 80 | 1.62 | 0.026 |
| | Polyvinylpyrrolidone | | | |
| 69 | 25 mg/mL | 5 mg/mL Polysorbate 20 | 1.56 | 0.016 |
| | Polyvinylpyrrolidone | | | |
| 70 | 25 mg/mL Sorbitol | 25 mg/mL Sorbitol | 1.45 | 0.029 |
| 71 | 25 mg/mL Sorbitol | 50 mg/mL Polysorbate 80 | 1.65 | 0.019 |
| 72 | 25 mg/mL Sorbitol | 5 mg/mL Polysorbate 20 | 1.95 | 0.052 |
| 73 | 3 mg/mL Chlorobutanol | 25 mg/mL Sorbitol | 0.79 | 0.055 |
| 74 | 3 mg/mL Chlorobutanol | 50 mg/mL PEG-35 castor oil | 1.22 | 0.061 |
| 75 | 3 mg/mL Chlorobutanol | 50 mg/mL Polysorbate 80 | 1.02 | 0.073 |
| 76 | 40 mg/mL Benzethonium | 20 mg/mL Calcium gluceptate | 1.00 | 0.041 |
| | chloride | | | |
| 77 | 40 mg/mL Benzethonium | 3 mg/mL Chlorobutanol | 1.00 | 0.001 |
| | chloride | | | |
| 78 | 40 mg/mL Benzethonium | 40 mg/mL Benzethonium | 1.07 | 0.019 |
| | chloride | chloride | | |
| 79 | 40 mg/mL Benzethonium | 50 mg/mL PEG-35 castor oil | 1.66 | 0.031 |
| | chloride | | | |
| 80 | 40 mg/mL Benzethonium | 50 mg/mL Polysorbate 80 | 0.61 | 0.013 |
| | chloride | | | |
| 81 | 40 mg/mL Diethanolamine | 0.6 mg/mL Lecithin | 2.10 | 0.006 |
| 82 | 40 mg/mL Diethanolamine | 14 mg/mL Gamma- | 1.84 | 0.022 |
| | | Cyclodextrin | | |
| 83 | 40 mg/mL Diethanolamine | 25 mg/mL Mannitol | 1.37 | 0.012 |
| 84 | 40 mg/mL Diethanolamine | 25 mg/mL | 0.86 | 0.176 |
| | | Polyvinylpyrrolidone | | |
| 85 | 40 mg/mL Diethanolamine | 25 mg/mL Sorbitol | 1.90 | 0.066 |
| 86 | 40 mg/mL Diethanolamine | 40 mg/mL Diethanolamine | 0.93 | 0.054 |
| 87 | 40 mg/mL Diethanolamine | 40 mg/mL Lysine | 2.07 | 0.003 |
| 88 | 40 mg/mL Diethanolamine | 40 mg/mL Sodium Acetate | 1.55 | 0.014 |
| 89 | 40 mg/mL Diethanolamine | 50 mg/mL Ethanol | 0.92 | 0.045 |
| 90 | 40 mg/mL Diethanolamine | 50 mg/mL Glycerin | 1.72 | 0.006 |
| 91 | 40 mg/mL Diethanolamine | 50 mg/mL Hydroxypropyl-b- | 1.26 | 0.04 |
| | | cyclodextrin | | |
| 92 | 40 mg/mL Diethanolamine | 50 mg/mL PEG-400 | 1.39 | 0.014 |
| 93 | 40 mg/mL Diethanolamine | 50 mg/mL PEG-6 | 0.48 | 0.266 |
| 94 | 40 mg/mL Diethanolamine | 50 mg/mL Poloxamer 188 | 1.66 | 0.016 |
| 95 | 40 mg/mL Diethanolamine | 50 mg/mL Polysorbate 80 | 1.89 | 0.013 |
| 96 | 40 mg/mL Diethanolamine | 50 mg/mL Propylene glycol | 1.25 | 0.008 |
| 97 | 40 mg/mL Diethanolamine | 5 mg/mL Polysorbate 20 | 1.82 | 0.049 |
| 98 | 40 mg/mL Lysine | 25 mg/mL | 0.64 | 0.036 |
| | | Polyvinylpyrrolidone | | |
| 99 | 40 mg/mL Lysine | 25 mg/mL Sorbitol | 2.39 | 0.01 |
| 100 | 40 mg/mL Lysine | 50 mg/mL Poloxamer 188 | 1.26 | 0.056 |
| 101 | 40 mg/mL Lysine | 50 mg/mL Polysorbate 80 | 1.47 | 0.023 |
| 102 | 40 mg/mL Lysine | 5 mg/mL Polysorbate 20 | 1.83 | 0.027 |
| 103 | 40 mg/mL Magnesium chloride | 25 mg/mL Sorbitol | 1.95 | 0.021 |
| 104 | 40 mg/mL Magnesium chloride | 50 mg/mL Poloxamer 188 | 1.32 | 0.043 |
| 105 | 40 mg/mL Magnesium chloride | 50 mg/mL Polysorbate 80 | 1.72 | 0.042 |
| 106 | 40 mg/mL Magnesium chloride | 5 mg/mL Polysorbate 20 | 2.42 | 0.112 |
| 107 | 40 mg/mL PEG-12 | 25 mg/mL | 0.46 | 0.051 |
| | | Polyvinylpyrrolidone | | |
| 108 | 40 mg/mL PEG-12 | 25 mg/mL Sorbitol | 2.15 | 0.027 |
| 109 | 40 mg/mL PEG-12 | 50 mg/mL Poloxamer 188 | 1.72 | 0.079 |
| 110 | 40 mg/mL PEG-12 | 50 mg/mL Polysorbate 80 | 1.74 | 0.027 |
| 111 | 40 mg/mL PEG-12 | 5 mg/mL Polysorbate 20 | 2.11 | 0.064 |
| 112 | 40 mg/mL Sodium Acetate | 25 mg/mL Sorbitol | 1.65 | 0.025 |
| 113 | 40 mg/mL Sodium Acetate | 50 mg/mL Polysorbate 80 | 1.60 | 0.015 |
| 114 | 40 mg/mL Sodium Acetate | 5 mg/mL Polysorbate 20 | 1.74 | 0.064 |
| 115 | 40 mg/mL Sodium benzoate | 25 mg/mL Sorbitol | 1.47 | 0.025 |
| 116 | 40 mg/mL Sodium benzoate | 40 mg/mL Sodium benzoate | 0.49 | 0.037 |
| 117 | 40 mg/mL Sodium benzoate | 50 mg/mL Polysorbate 80 | 2.27 | 0.071 |
| 118 | 40 mg/mL Sodium benzoate | 5 mg/mL Polysorbate 20 | 3.95 | 0.19 |
| 119 | 40 mg/mL Sodium tartrate | 25 mg/mL Sorbitol | 1.76 | 0.049 |
| 120 | 40 mg/mL Sodium tartrate | 50 mg/mL Polysorbate 80 | 1.94 | 0.055 |
| 121 | 40 mg/mL Sodium tartrate | 5 mg/mL Polysorbate 20 | 1.85 | 0.011 |
| 122 | 50 mg/mL Ethanol | 25 mg/mL Sorbitol | 1.59 | 0.006 |
| 123 | 50 mg/mL Ethanol | 50 mg/mL Glycerin | 1.55 | 0.03 |
| 124 | 50 mg/mL Ethanol | 50 mg/mL Hydroxypropyl-b- | 0.60 | 0.168 |
| | | cyclodextrin | | |
| 125 | 50 mg/mL Ethanol | 50 mg/mL Poloxamer 188 | 2.20 | 0.052 |
| 126 | 50 mg/mL Ethanol | 50 mg/mL Polysorbate 80 | 1.59 | 0.024 |
| 127 | 50 mg/mL Ethanol | 50 mg/mL Propylene glycol | 1.00 | 0.05 |
| 128 | 50 mg/mL Ethanol | 5 mg/mL Polysorbate 20 | 1.75 | 0.015 |
| 129 | 50 mg/mL Glycerin | 0.6 mg/mL Lecithin | 0.60 | 0.039 |
| 130 | 50 mg/mL Glycerin | 25 mg/mL | 0.64 | 0.024 |
| | | Polyvinylpyrrolidone | | |
| 131 | 50 mg/mL Glycerin | 25 mg/mL Sorbitol | 1.72 | 0.031 |
| 132 | 50 mg/mL Glycerin | 50 mg/mL Hydroxypropyl-b- | 0.41 | 0.006 |
| | | cyclodextrin | | |
| 133 | 50 mg/mL Glycerin | 50 mg/mL Poloxamer 188 | 0.96 | 0.046 |
| 134 | 50 mg/mL Glycerin | 50 mg/mL Polysorbate 80 | 1.55 | 0.005 |
| 135 | 50 mg/mL Glycerin | 5 mg/mL Polysorbate 20 | 1.87 | 0.04 |
| 136 | 50 mg/mL Hydroxypropyl-b- | 1.0 mg/mL Methylparaben | 0.47 | 0.009 |
| | cyclodextrin | | | |
| 137 | 50 mg/mL Hydroxypropyl-b- | 25 mg/mL Mannitol | 0.57 | 0.02 |
| | cyclodextrin | | | |
| 138 | 50 mg/mL Hydroxypropyl-b- | 25 mg/mL | 0.72 | 0.015 |
| | cyclodextrin | Polyvinylpyrrolidone | | |
| 139 | 50 mg/mL Hydroxypropyl-b- | 25 mg/mL Sorbitol | 1.47 | 0.029 |
| | cyclodextrin | | | |
| 140 | 50 mg/mL Hydroxypropyl-b- | 40 mg/mL PEG-12 | 0.43 | 0.011 |
| | cyclodextrin | | | |
| 141 | 50 mg/mL Hydroxypropyl-b- | 40 mg/mL Sodium Acetate | 0.50 | 0.026 |
| | cyclodextrin | | | |
| 142 | 50 mg/mL Hydroxypropyl-b- | 40 mg/mL Sodium benzoate | 0.51 | 0.026 |
| | cyclodextrin | | | |
| 143 | 50 mg/mL Hydroxypropyl-b- | 40 mg/mL Sodium tartrate | 0.54 | 0.028 |
| | cyclodextrin | | | |
| 144 | 50 mg/mL Hydroxypropyl-b- | 50 mg/mL Poloxamer 188 | 1.10 | 0.02 |
| | cyclodextrin | | | |
| 145 | 50 mg/mL Hydroxypropyl-b- | 50 mg/mL Polysorbate 80 | 1.90 | 0.022 |
| | cyclodextrin | | | |
| 146 | 50 mg/mL Hydroxypropyl-b- | 5 mg/mL Polysorbate 20 | 1.97 | 0.035 |
| | cyclodextrin | | | |
| 147 | 50 mg/mL PEG-35 castor oil | 0.6 mg/mL Lecithin | 1.06 | 0.041 |
| 148 | 50 mg/mL PEG-35 castor oil | 1.0 mg/mL Methylparaben | 0.69 | 0.021 |
| 149 | 50 mg/mL PEG-35 castor oil | 14 mg/mL Gamma- | 1.06 | 0.026 |
| | | Cyclodextrin | | |
| 150 | 50 mg/mL PEG-35 castor oil | 25 mg/mL Deoxycholic Acid | 0.71 | 0.048 |
| 151 | 50 mg/mL PEG-35 castor oil | 25 mg/mL Mannitol | 0.59 | 0.007 |
| 152 | 50 mg/mL PEG-35 castor oil | 25 mg/mL | 0.82 | 0.038 |
| | | Polyvinylpyrrolidone | | |
| 153 | 50 mg/mL PEG-35 castor oil | 25 mg/mL Sorbitol | 1.15 | 0.021 |
| 154 | 50 mg/mL PEG-35 castor oil | 40 mg/mL Diethanolamine | 1.17 | 0.02 |
| 155 | 50 mg/mL PEG-35 castor oil | 40 mg/mL Lysine | 0.94 | 0.036 |
| 156 | 50 mg/mL PEG-35 castor oil | 40 mg/mL Magnesium | 0.72 | 0.08 |
| | | chloride | | |
| 157 | 50 mg/mL PEG-35 castor oil | 40 mg/mL PEG-12 | 0.83 | 0.023 |
| 158 | 50 mg/mL PEG-35 castor oil | 40 mg/mL Sodium Acetate | 0.96 | 0.042 |
| 159 | 50 mg/mL PEG-35 castor oil | 40 mg/mL Sodium benzoate | 0.91 | 0.021 |
| 160 | 50 mg/mL PEG-35 castor oil | 40 mg/mL Sodium tartrate | 1.13 | 0.05 |
| 161 | 50 mg/mL PEG-35 castor oil | 50 mg/mL Ethanol | 1.26 | 0.021 |
| 162 | 50 mg/mL PEG-35 castor oil | 50 mg/mL Glycerin | 0.99 | 0.032 |
| 163 | 50 mg/mL PEG-35 castor oil | 50 mg/mL Hydroxypropyl-b- | 0.93 | 0.017 |
| | | cyclodextrin | | |
| 164 | 50 mg/mL PEG-35 castor oil | 50 mg/mL PEG-400 | 0.72 | 0.009 |
| 165 | 50 mg/mL PEG-35 castor oil | 50 mg/mL PEG-6 | 0.66 | 0.037 |
| 166 | 50 mg/mL PEG-35 castor oil | 50 mg/mL Poloxamer 188 | 0.66 | 0.039 |
| 167 | 50 mg/mL PEG-35 castor oil | 50 mg/mL Polysorbate 80 | 2.59 | 0.081 |
| 168 | 50 mg/mL PEG-35 castor oil | 50 mg/mL Propylene glycol | 0.68 | 0.025 |
| 169 | 50 mg/mL PEG-35 castor oil | 5 mg/mL Polysorbate 20 | 3.02 | 0.107 |
| 170 | 50 mg/mL PEG-400 | 25 mg/mL Sorbitol | 2.11 | 0.05 |
| 171 | 50 mg/mL PEG-400 | 50 mg/mL Poloxamer 188 | 0.79 | 0.062 |
| 172 | 50 mg/mL PEG-400 | 50 mg/mL Polysorbate 80 | 2.12 | 0.085 |
| 173 | 50 mg/mL PEG-400 | 5 mg/mL Polysorbate 20 | 2.17 | 0.084 |
| 174 | 50 mg/mL PEG-6 | 25 mg/mL Sorbitol | 2.45 | 0.021 |
| 175 | 50 mg/mL PEG-6 | 50 mg/mL Poloxamer 188 | 0.58 | 0.043 |
| 176 | 50 mg/mL PEG-6 | 50 mg/mL Polysorbate 80 | 2.60 | 0.028 |
| 177 | 50 mg/mL PEG-6 | 5 mg/mL Polysorbate 20 | 2.12 | 0.055 |
| 178 | 50 mg/mL Poloxamer 188 | 25 mg/mL | 0.83 | 0.031 |
| | | Polyvinylpyrrolidone | | |
| 179 | 50 mg/mL Poloxamer 188 | 25 mg/mL Sorbitol | 2.41 | 0.029 |
| 228 | 50 mg/mL Ethanol | 40 mg/mL Sodium Acetate | 0.44 | 0.008 |
| 229 | 50 mg/mL Hydroxypropyl-b- | 40 mg/mL Lysine | 0.45 | 0.033 |
| | cyclodextrin | | | |
| 230 | 50 mg/mL Hydroxypropyl-b- | 40 mg/mL Magnesium | 0.46 | 0.017 |
| | cyclodextrin | chloride | | |
| 231 | 50 mg/mL Hydroxypropyl-b- | 50 mg/mL PEG-400 | 0.56 | 0.022 |
| | cyclodextrin | | | |
| 232 | 50 mg/mL Hydroxypropyl-b- | 50 mg/mL PEG-6 | 0.51 | 0.023 |
| | cyclodextrin | | | |
| 233 | 50 mg/mL Hydroxypropyl-b- | 50 mg/mL Propylene glycol | 0.40 | 0.069 |
| | cyclodextrin | | | |
| 234 | 50 mg/mL Propylene glycol | 25 mg/mL | 0.44 | 0.057 |
| | | Polyvinylpyrrolidone | | |
| 235 | 0.6 mg/mL Lecithin | 1.0 mg/mL Methylparaben | 0.51 | 0.014 |
| 236 | 0.6 mg/mL Lecithin | 25 mg/mL Mannitol | 0.40 | 0.033 |
| 237 | 0.6 mg/mL Lecithin | 40 mg/mL Sodium Acetate | 0.59 | 0.013 |
| 238 | 0.6 mg/mL Lecithin | 40 mg/mL Sodium benzoate | 0.40 | 0.021 |
| 239 | 0.6 mg/mL Lecithin | 40 mg/mL Sodium tartrate | 0.58 | 0.052 |
| 240 | 0.6 mg/mL Lecithin | 50 mg/mL PEG-6 | 0.63 | 0.032 |
| 241 | 0.6 mg/mL Lecithin | 50 mg/mL Propylene glycol | 0.70 | 0.052 |
| 242 | 1.0 mg/mL Methylparaben | 40 mg/mL PEG-12 | 0.43 | 0.021 |
| 243 | 1.0 mg/mL Methylparaben | 50 mg/mL PEG-6 | 0.42 | 0.014 |
| 244 | 1.0 mg/mL Methylparaben | 50 mg/mL Poloxamer 188 | 1.99 | 0.027 |
| 245 | 14 mg/mL Gamma- | 1.0 mg/mL Methylparaben | 0.59 | 0.019 |
| | Cyclodextrin | | | |
| 246 | 14 mg/mL Gamma- | 40 mg/mL PEG-12 | 0.49 | 0.026 |
| | Cyclodextrin | | | |
| 247 | 20 mg/mL Calcium gluceptate | 50 mg/mL Ethanol | 0.58 | 0.048 |
| 248 | 25 mg/mL Lactose | 0.6 mg/mL Lecithin | 0.47 | 0.051 |
| 249 | 25 mg/mL Lactose | 40 mg/mL Lysine | 0.66 | 0.065 |
| 250 | 25 mg/mL Lactose | 40 mg/mL Sodium benzoate | 0.55 | 0.02 |
| 251 | 25 mg/mL Lactose | 40 mg/mL Sodium tartrate | 0.46 | 0.013 |
| 252 | 25 mg/mL Lactose | 50 mg/mL Glycerin | 0.78 | 0.018 |
| 253 | 25 mg/mL Lactose | 50 mg/mL PEG-6 | 0.48 | 0.058 |
| 254 | 25 mg/mL Mannitol | 1.0 mg/mL Methylparaben | 0.59 | 0.084 |
| 255 | 25 mg/mL Mannitol | 50 mg/mL PEG-400 | 0.46 | 0.015 |
| 256 | 25 mg/mL Mannitol | 50 mg/mL Propylene glycol | 0.66 | 0.048 |
| 257 | 3 mg/mL Chlorobutanol | 0.6 mg/mL Lecithin | 0.41 | 0.03 |
| 258 | 3 mg/mL Chlorobutanol | 40 mg/mL Diethanolamine | 0.60 | 0.006 |
| 259 | 40 mg/mL Benzethonium | 40 mg/mL Sodium benzoate | 0.44 | 0.032 |
| | chloride | | | |
| 260 | 40 mg/mL Lysine | 1.0 mg/mL Methylparaben | 0.62 | 0.022 |
| 261 | 40 mg/mL Lysine | 25 mg/mL Mannitol | 0.77 | 0.027 |
| 262 | 40 mg/mL Lysine | 40 mg/mL Lysine | 0.78 | 0.033 |
| 263 | 40 mg/mL Lysine | 50 mg/mL PEG-6 | 0.55 | 0.047 |
| 264 | 40 mg/mL Magnesium chloride | 40 mg/mL Magnesium | 0.45 | 0.018 |
| | | chloride | | |
| 265 | 40 mg/mL Magnesium chloride | 40 mg/mL Sodium Acetate | 0.46 | 0.025 |
| 266 | 40 mg/mL Magnesium chloride | 50 mg/mL PEG-400 | 0.42 | 0.022 |
| 267 | 40 mg/mL Sodium tartrate | 40 mg/mL Sodium tartrate | 0.41 | 0.013 |
| 268 | 50 mg/mL Ethanol | 1.0 mg/mL Methylparaben | 0.71 | 0.015 |
| 269 | 50 mg/mL Ethanol | 25 mg/mL Mannitol | 0.54 | 0.013 |
| 270 | 50 mg/mL Ethanol | 40 mg/mL Sodium benzoate | 1.00 | 0.015 |
| 271 | 50 mg/mL Ethanol | 50 mg/mL PEG-400 | 0.53 | 0.001 |
| 272 | 50 mg/mL Ethanol | 50 mg/mL PEG-6 | 5.34 | 0.906 |
| 273 | 50 mg/mL Hydroxypropyl-b- | 0.6 mg/mL Lecithin | 0.71 | 0.011 |
| | cyclodextrin | | | |
| 274 | 50 mg/mL PEG-35 castor oil | 50 mg/mL PEG-35 castor oil | 1.23 | 0.073 |
| 275 | 50 mg/mL Propylene glycol | 40 mg/mL Sodium Acetate | 0.50 | 0.022 |

### EXAMPLE2

Infusion Formulations

The following five two-excipient system lansoprazole formulations were made in accordance with the present invention. In each of these formulations, the excipients are present in the formulation in an approximate 1:1 volumetric ratio that does not take into account the fact that lansoprazole powder was added to the formulations after the excipients were mixed. The five formulations are identified below in Table 3.

TABLE 3

| Formulation | API (mg/mL) | Excipients |
|---|---|---|
| 1 | ≦30 | Polysorbate 80 |
| | | PEG 400 |
| 2 | ≦30 | Polysorbate 80 |
| | | Polypropylene Glycol(PPG) |
| 3 | ≦25 | Polysorbate 80 |
| | | Ethanol |
| 4 | ≦25 | PEG 300 |
| | | Polypropylene Glycol (PPG) |
| 5 | ≦20 | Polysorbate 20 |
| | | PEG 300 (PEG) |

### EXAMPLE 3

Injectable Formulations

Injectable formulations of the present invention were made that comprised lansoprazole at a concentration of from about 30 mg/mL to greater than 40 mg/mL. A preferred injectable formulation is shown below in Table 4, formulation 4. This formulation comprises four excipients: Polysorbate 80, polypropylene glycol, PEG 300, and ethanol in a volumetric ratio of 2:1:0.8:0.2. The formulations shown may optionally further contain preservatives and diluents. The volumetric ratios of the various pure excipients are listed in Table 4. All of the formulations listed in Table 4 proved stable for an extended period of eight to ten hours at room temperature.

TABLE 4

| Formu- | API | | |
|---|---|---|---|
| lation | (mg/mL) | Excipients | Volumetric Ratio |
| 1 | <35 | Polysorbate 80 | 3:1 Polysorbate:PPG |
| | | Polypropylene | |
| | | Glycol(PPG) | |
| 2 | ≦35 | Polysorbate 80 | 0.3:0.8:0.2 |
| | | Polypropylene | Polysorbate:PPG:Ethanol |
| | | Glycol(PPG) | |
| | | Ethanol | |
| 3 | ≦35 | Polysorbate 80 | 2.5:1.0:0.5 |
| | | Polypropylene | Polysorbate:PPG:PEG 300 |
| | | Glycol(PPG) | |
| | | PEG 300 | |
| 4 | ≧40 | Polysorbate 80 | 2.0:1.0:0.8:0.2 |
| | | Polypropylene | Polysorbate:PPG:PEG 300:Ethanol |
| | | Glycol(PPG) | |
| | | PEG 300 | |
| | | Ethanol | |

While the high concentration of lansoprazole in formulation 4 (Table 4) proves desirable for storage purposes given the relative instability of lansoprazole, the formulation preferably is diluted by about ten-fold for use in a bolus injection at a lansoprazole concentration of about 4 mg/mL.

1-84. (canceled)

85. A liquid lansoprazole formulation comprising lansoprazole and an excipient system, wherein:
(a) the concentration of lansoprazole in the formulation ranges from about 0.3 mg/mL to about 50 mg/mL;
(b) the excipient system comprises either a single excipient, or a combination of two to four compositionally distinct excipients, wherein each excipient is'selected from the group of excipient categories consisting of: a hydrotrope, a preservative, a pharmaceutically acceptable salt, a surfactant, a base, a cyclodextrin, a viscosity modifier, an emulsifier, a solvent, a carrier, and a lubricant; and
(c) the formulation may be administered parenterally to a mammal in need thereof.

86. The formulation of claim 85, comprising a two-excipient system in which the concentration of each excipient in the formulation ranges from about 0.4 mg/mL to about 60 mg/mL and total excipient concentration in the formulation ranges from about 0.8 mg/mL to about 120 mg/mL.

87. The formulation of claim 86, further comprising a diluent and a preservative that acts as an antimicrobial agent.

88. The formulation of claim 85, comprising lansoprazole and a single excipient.

89. The formulation of claim 85, comprising lansoprazole in combination with a two-excipient system comprising either compositionally distinct first and second excipients selected from the same excipient category, or compositionally distinct first and second excipients selected from different excipient categories.

90. The formulation of claim 88, wherein the excipient is either a hydrotrope, a preservative, a pharmaceutically acceptable salt, a surfactant, a base, a cyclodextrin, a viscosity modifier, an emulsifier, a solvent, a carrier, or a lubricant.

91. The formulation of claim 89, wherein the excipient system comprises either compositionally distinct first and second emulsifiers, or an emulsifier in combination with: (i) a viscosity modifier, (ii) a carrier, (iii) a base, (iv) a solvent, or (v) a surfactant.

92. The formulation of claim 89, wherein the excipient system comprises a preservative in combination with: (i) a carrier, (ii) a surfactant, (iii) a solvent, or (iv) a cyclodextrin.

93. The formulation of claim 89, wherein the excipient system comprises either compositionally distinct first and second cyclodextrins, or a cyclodextrin in combination with: (i) an emulsifier, (ii) a viscosity modifier, (iii) a carrier, (iv) a lubricant, (v) a surfactant, or (vi) a solvent.

94. The formulation of claim 89, wherein the excipient system comprises a pharmaceutically acceptable calcium salt in combination with: (i) a carrier, (ii) a base, (iii) a solvent, or (iv) a surfactant.

95. The formulation of claim 89, wherein the excipient system comprises either compositionally distinct first and second surfactants, or a surfactant in combination with: (i) a carrier, (ii) a viscosity modifier, (iii) a base, (iv) a pharmaceutically acceptable salt other than a calcium salt, (v) a solvent, (vi) a lubricant, or (vii) a hydrotrope.

96. The formulation of claim 89, wherein the excipient system comprises a hydrotrope in combination with: (i) a viscosity modifier, (ii) a carrier, (iii) a preservative, (iv) a base, (v) a solvent, or (vi) a cyclodextrin.

97. The formulation of claim 89, wherein the excipient system comprises either compositionally distinct first and second viscosity modifiers, or a viscosity modifier in combination with: (i) a carrier, (ii) a lubricant, or (iii) a solvent.

98. The formulation of claim 89, wherein the excipient system comprises either compositionally distinct first and second carriers, or a carrier in combination with: (i) a solvent, or (ii) a lubricant.

99. The formulation of claim 89, wherein the excipient system comprises either compositionally distinct first and second bases, or a base in combination with: (i) a preservative, (ii) a solvent, (iii) a carrier, (iv) a viscosity modifier, (v) a lubricant, or (vi) a cyclodextrin.

100. The formulation of claim 89, wherein the excipient system comprises either compositionally distinct first and second solvents, or a solvent in combination with: (i) a salt, or (ii) a lubricant.

101. The formulation of claim 89, wherein the excipient system comprises compositionally distinct first and second lubricants.

102. The formulation of claim 88, wherein the excipient is selected from the group consisting of: sorbitol, mannitol, lactose, benzethonium chloride, chlorobutanol, methylparaben, methanol, ethanol, i-propanol, n-butanol, calcium chloride, magnesium chloride, calcium gluconate, calcium glubionate, calcium gluceptate, polyoxyethylated castor oil, polysorbate 20, polysorbate 80, poloxamers, deoxycholic acid and salts of deoxycholic acid, lysine, diethanolamine, gamma cyclodextrin, hydroxypropyl-beta-cyclodextrin, polyvinylpyrrolidone, glycerin, lecithin, sodium benzoate, sodium acetate, sodium tartrate, polyethylene glycol, starch, and propylene glycol.

103. The formulation of claim 89, wherein each excipient is selected from the group consisting of: sorbitol, mannitol, lactose, benzethonium chloride, chlorobutanol, methylparaben, methanol, ethanol, i-propanol, n-butanol, calcium chloride, magnesium chloride, calcium gluconate, calcium glubionate, calcium gluceptate, polyoxyethylated castor oil , polysorbate 20, polysorbate 80, poloxamers, deoxycholic acid and salts of deoxycholic acid, lysine, diethanolamine, gamma cyclodextrin, hydroxypropyl-beta-cyclodextrin, polyvinylpyrrolidone, glycerin, lecithin, sodium benzoate, sodium acetate, sodium tartrate, polyethylene glycol, starch, and propylene glycol.

104. The formulation of claim 85, wherein:
(a) the concentration of lansoprazole in the formulation is between about 4.0 mg/mL to 50 mg/mL; and
(b) the excipient system is a four-excipient system comprising a surfactant, compositionally distinct first and second solvents, and an alcohol.

105. The formulation of claim 104, wherein the surfactant is a polysorbate, the first and second solvents are polyethylene glycols, and the alcohol is methanol, ethanol, i-propanol or n-butanol.

106. The formulation of claim 105, wherein the surfactant is polysorbate 80, the first or second solvent is PEG-300, and the alcohol is ethanol.

107. The formulation of claim 85, wherein:
(a) the concentration of lansoprazole in the formulation is between about 0.4 mg/mL to about 40 mg/mL; and
(b) the excipient system is a three-excipient system comprising a surfactant and compositionally distinct first and second solvents.

108. The formulation of claim 107, wherein the surfactant is a polysorbate and the first and second solvents are compositionally distinct polyethylene glycols.

109. The formulation of claim 108, wherein the surfactant is polysorbate 80 and the first or second solvent is PEG-300.
